(19) United States
(12) Patent Application Publication
(10) Pub. No.: US 2008/0275131 A1
(43) Pub. Date: Nov. 6, 2008

(54) COMPOSITIONS OF (-)-E-10-OH-NT AND METHODS FOR THEIR SYNTHESIS AND USE

(75) Inventors: Bertrand LE BOURDONNEC, East Fallowfield, PA (US); Roland E. DOLLE, King of Prussia, PA (US)

Correspondence Address: DECHERT LLP, P.O. BOX 390460, MOUNTAIN VIEW, CA, 94039-0460, US

(73) Assignee: Adolor Corporation, Exton, PA (US)

(21) Appl. No.: 12/107,742

(22) Filed: Apr. 22, 2008

Related U.S. Application Data
(60) Provisional application No. 60/915,103, filed on Apr. 30, 2007.
(60) Provisional application No. 61/027,814, filed on Feb. 11, 2008.
(60) Provisional application No. 61/028,122, filed on Feb. 12, 2008.

(51) Int. Cl. A61K 31/135; C07C 211/31; A61P 29/00; A61P 25/24

(52) U.S. Cl. 514/649; 564/336

(57) ABSTRACT

This present disclosure provides compositions comprising E-10-OH-NT metabolites of AT and NT, methods for their synthesis and methods for their use.

### 1. CROSS REFERENCE TO RELATED APPLICATIONS

This application claims priority under 35 U.S.C. § 1.119(e) to provisional application No. 60/915,103 filed Apr. 30, 2007, provisional application No. 61/027,814 filed Feb. 11, 2008, and provisional application No. 61/028,122 filed Feb. 12, 2008, the disclosures of which are incorporated herein by reference in their entireties.

### 2. STATEMENT REGARDING FEDERALLY SPONSORED RESEARCH

None.

### 3. PARTIES TO A JOINT RESEARCH AGREEMENT

None.

### 4. REFERENCE TO SEQUENCE LISTING, TABLE OR COMPUTER PROGRAM

None.

### 5. BACKGROUND

Tricyclic antidepressants, including amitriptyline (AT) and nortryptiline (NT), have been shown to inhibit the reuptake of monoamines, including norepinephrine and serotonin, and have been widely used both on and off label to treat many different disorders and diseases mediated, at least in part, by dysregulated uptake or reuptake of norepinephrine and serotonin, including, but not limited to, mood disorders such as depression, anxiety disorders such as obsessive compulsive disorder (OCD), eating disorders such as anorexia nervosa and bulimia nervosa, impulse-control disorders such as tricholtillomania, sleep disorders such as insomnia related to opioid withdrawal, personality disorders such as attention deficit hyperactivity disorder (ADHD) and somatoform disorders such as certain types of pain. AT and NT have also been used as first-line treatment for various types of acute and chronic pain that are either nociceptive (for example somatic or visceral) or non-nociceptive (for example neuropathic or sympathetic) in origin, including non-nociceptive neuropathic pains such as diabetic neuropathy and post herpetic neuralgia (PHN), and nociceptive pain including inflammatory pain and interstitial cystitis.

However, the use of AT and NT has been limited by their untoward side effects, which include, but are not limited to, antimuscarinic effects such as dry mouth, constipation, nausea and urinary retention; headaches, increased sweating, tinnitus, unpleasant taste, cardiotoxic effects such as orthostatic hypotension, arrhythmias, and tachycardia; sedation and weight gain. NT and AT are also contraindicated for use with various mediations due to shared side effects and/or the interaction of NT and AT with the cytochrome P450 drug metabolizing enzymes.

Consequentially, identification of other compounds capable of treating disorders and diseases mediated, at least in part, by dysregulated uptake or reuptake of norepinephrine and serotonin and other targets that have been implicated in the pharmacology of AT and tricyclic antidepressants, but which exhibit reduced and/or fewer side effects and which can be administered with drugs which are contraindicated for use with AT and NT would be beneficial and desirable.

### 6. SUMMARY

When administered to mammals, including humans, in vivo, amitriptyline (“AT”) and nortriptyline (“NT”) share a number of common metabolites, the major one of which is [(5-[3-methylamino-propylidene]-10,11-dihydro-5H-dibenzo[a,d]cyclohepten-10-ol] (“10-OH-NT”). This 10-OH-NT metabolite has two geometric isomers depending upon the configuration about the double bond (Z-10-OH-NT and E-10-OH-NT). Each of these geometric isomers comprises two enantiomers, (+)-E-10-OH-NT, and (−)-E-10-OH-NT, and (+)-Z-10-OH-NT and (−)-Z-10-OH-NT, owing to chirality at the carbon at the 10-position. Certain studies suggest that the racemate of the major 10-OH-NT metabolite, (±)-E-10-OH-NT, might be useful to treat depression and anxiety when administered orally to humans (Nordin et al., 1987a). However, with the exception of a mention to an unpublished study stating that the (+) and (−) enantiomers of E-10-OH-NT inhibit noradreneline uptake with similar potency (Nordin & Bertilsson, 1995), the specific pharmacological and biochemical properties of this racemate, and also the isolated (+) and (−) enantiomers, have not been reported in the literature.

Compelling evidence discovered by the applicants and presented herein (discussed in more detail in later sections) demonstrates that the (−) enantiomer of E-10-OH-NT possesses important pharmacological and biochemical properties consistent with therapeutic usefulness. For example, like AT and NT, (−)-E-10-OH-NT binds norepinephrine (“NE”) and serotonin (“5HT”) monoamine transporters and inhibits uptake of NE and 5HT, biological activities known to be associated with the antidepressant and other therapeutic properties of AT and NT. Significantly, the affinity of (−)-E-10-OH-NT for the NE transporter, as measured in a recombinant transporter binding assay, was approximately equal to that of AT, and within about 10-fold that of NT. Similar results were observed for inhibition of NE uptake as measured in a cell-based functional assay. Moreover, like NT, (−)-E-10-OH-NT exhibited selectivity for the NE transporter versus the 5HT transporter.

The applicants have discovered that (−)-E-10-OH-NT does not share in common with AT and NT certain pharmacological and biochemical properties that correlate with untoward side effects during use. For example, while both AT and NT potently antagonize the H₁ histaminergic receptor, a property that plays a role in the significant weight gain (Altamura et al., 1989; Wirshing et al., 1999; Richelson, 2001; Khawam et al., 2006) and undesirable sedative effects (Bryson & Wilde, 1996) observed with both short and long term use of AT and NT, (−)-E-10-OH-NT antagonizes the H₁ histaminergic receptor to a much lesser extent. Indeed, the superior non-sedating effects of (−)-E-10-OH-NT as compared to AT and NT were confirmed in a rat rotarod assay commonly used to assess the sedative properties of drugs. As a consequence, (−)-E-10-OH-NT should be less sedating and have fewer adverse appetite effects than AT and NT. In this assay, peak deficits on rotarod performance of 51±20% and 60±40% were observed at 30 minutes following administration of AT (30 mg/kg, i.p.) and NT (30 mg/kg, i.p.), respectively. In stark contrast, no significant impairment in performance was observed in rats receiving (−)-E-10-OH-NT at the dose of 30 mg/kg, i.p.

These and other newly discovered beneficial properties of (−)-E-10-OH-NT (discussed further below) offer therapeutic benefits that are unprecedented in the art for tricyclic antidepressant drugs. Because (−)-E-10-OH-NT shares therapeutically relevant pharmacological and biochemical properties with AT and NT, it can be used, either in substantially enantiomerically pure or enantiomerically pure form to treat the many indications commonly treated with AT and NT, including but not limited to the various diseases and disorders mentioned further herein. However, because (−)-E-10-OH-NT does not possess certain pharmacological and biochemical properties known to be responsible for untoward side effects, therapy can be administered to patients with a lower incidence of undesirable side effects. As a specific, non-limiting example, because (−)-E-10-OH-NT is a significantly less potent H₁ histaminergic receptor antagonist than AT and NT, therapeutic benefit can be achieved with this agent without the undesirable side effects of significant weight gain and sedation experienced with AT and/or NT therapy.

Like (−)-E-10-OH-NT, racemic (±)-E-10-OH-NT and its (+) enantiomer also possess important and approximately equipotent biochemical activities in in vitro assays. For example, (±)-E-10-OH-NT, (+)-E-10-OH-NT and (−)-E-10-OH-NT each exhibit approximately equal affinities for the norepinephrine transporter and also the serotonin transporter (see Example 14). In functional assays, the (+) and (−) enantiomers of E-10-OH-NT exhibit approximately equipotent selective inhibition of norepinephrine uptake, and are approximately equipotent to AT in functional in vitro assays (see Example 5). Yet, in vivo animal studies indicate that the (−)-E-10-OH NT enantiomer is therapeutically more effective than both racemic (±)-E-10-OH-NT and (+)-E-10-OH-NT in a rodent model of neuropathic pain (L5 Spinal Nerve Ligation rat model; see Example 15) and is also more effective than (+)-E-10-OH-NT in rodent models of hyperalgesia (FCA-induced inflammatory pain; see Examples 5 and 17-20) and depression (Forced Swim Test; see Example 21). Given the approximately equipotent biochemical activities of (±)-E-10-OH-NT, (+)-E-10-OH-NT and (−)-E-10-OH-NT, these in vivo results were quite surprising. It was expected that racemic (±)-E-10-OH-NT, (+)-E-10-OH-NT and (−)-E-10-OH-NT would exhibit approximately equipotent efficacies in in vivo rodent models of pain and depression. Yet, only the (−) enantiomer proved effective. The instant disclosure is based, in part, on the surprising efficacy discovered for (−)-E-10-OH-NT.

Accordingly, in one aspect, the present disclosure provides compositions comprising E-10-OH-NT and optionally one or more pharmaceutically acceptable carriers, excipients or diluents. The E-10-OH-NT is present in the composition as non-racemic mixture enriched in the (−) enantiomer. In some embodiments, the E-10-OH-NT comprising the composition is substantially enantiomerically pure (−)-E-10-OH-NT. In some embodiments, the E-10-OH-NT comprising the composition is enantiomerically pure (−)-E-10-OH-NT.

The E-10-OH-NT can be present in the composition in the form of the free base, or in the form of a salt. In some embodiments, the E-10-OH-NT is present in the form of a pharmaceutically acceptable acid addition salt. The E-10-OH-NT (including salt forms) can also be present in the composition in the form of a solvate and/or hydrate, for example with solvents and/or water used during preparation or purification.

The E-10-OH-NT composition can be used in vitro or in vivo, as will be described in more detail below. When used in vivo, the composition can be formulated for administration to animals in veterinary contexts, or for administration to humans via virtually any route or mode of administration, including but not limited to, oral, topical, ocular, buccal, systemic, nasal, injection, transdermal, rectal, vaginal, inhalation or insufflation.

As mentioned above, (−)-E-10-OH-NT shares important biological properties with AT and NT that correlate with the therapeutic efficacy of these drugs. It also exhibits efficacy in animal models of disease. Based upon these similar properties, it is expected that the compositions described herein will be equally effective at treating the numerous diseases and indications responsive to treatment with AT and NT. Thus, in another aspect, the present disclosure provides methods of treating diseases or indications responsive to AT or NT therapy. The methods generally comprise administering to a mammal, including a human, suffering from a disease or indication responsive to treatment with AT or NT, an amount of a composition described herein effective to treat the disease or indication. In some embodiments, the E-10-OH-NT composition comprises E-10-OH-NT that is enriched in the (−) enantiomer. In some embodiments, the E-10-OH-NT composition comprises substantially enantiomerically pure (−)-E-10-OH-NT. In some embodiments the E-10-OH-NT composition comprises enantiomerically pure (−)-E-10-OH-NT.

One important class of diseases or indications known to be responsive to treatment with AT and/or NT is mental disease. Specific examples of such mental diseases or indications include, but are not limited to, the various mental diseases and indications classified in the Diagnostic and Statistic Manual of Mental Disorders IV (Text Revision 2000; referred to hereinafter as “DSM-IV”) as mood disorders (such as, for example, depression), anxiety disorders (such as, for example OCD), eating disorders, (such as, for example, anorexia nervosa and bulimia nervosa), impulse disorders (such as, for example, trichotillomania), sleep disorders (such as, for example, insomnia related to opioid withdrawal), personality disorders (such as, for example, ADHD), and somatoform disorders (such as certain types of pain).

Another important class of diseases or indications known to be responsive to treatment with AT and/or NT is pain, including both acute and chronic pain, whether nociceptive (for example somatic or visceral) or non-nociceptive (for example neuropathic or sympathetic) in origin (discussed further below).

All of these diseases or indications are expected to respond to treatment with various embodiments of the E-10-OH-NT compositions described herein. And, as mentioned above, owing to the unexpected low antagonistic properties of (−)-E-10-OH-NT at the H₁ histaminergic receptor and other unexpected and unappreciated properties discussed further below, it is expected that treatment can be achieved with fewer untoward side effects than treatment with AT and/or NT.

When used to treat these various diseases or indications, the E-10-OH-NT compositions can be used alone as monotherapy, or, alternatively, they can be used in combination with, or adjunctively to, other treatments. For example, when used to treat specific mental diseases or indications, the E-10-OH-NT composition may be administered in combination with, or adjunctively to, another therapeutic agent useful to treat the same mental disease or indication. When used to treat specific types of pain, the E-10-OH-NT composition may be administered in combination with, or adjunctively to, another therapeutic agent useful to treat the same type of pain. However, such combination or adjunctive therapies are not limited to combinations of compounds useful to treat the same indication. In some embodiments it may be useful or desirable to administer the E-10-OH-NT composition in combination with, or adjunctively to, therapeutic agents that do not treat the disease or disorder being treated with the E-10-OH-NT composition. In some embodiments, the E-10-OH-NT composition to be administered in combination with, or adjunctively to, other treatments, comprises E-10-OH-NT that is enriched in the (−) enantiomer. In some embodiments, the E-10-OH-NT composition comprises substantially enantiomerically pure (−)-E-10-OH-NT. In some embodiments the E-10-OH-NT composition comprises enantiomerically pure (−)-E-10-OH-NT. Representative non-limiting examples of suitable combinations are discussed in more detail in a later section.

While not intending to be bound by any theory of operation, it is believed that the ability of AT and NT to inhibit monoamine transporters such as the NE and/or 5HT transporters is, in part, responsible for their many therapeutic properties. It is well precedented in the art that at least the following diseases or indications respond to treatment with inhibitors of NE and/or 5HT transporters: urinary disorders such as urinary incontinence; mood disorders such as depression and seasonal affective disorder (SAD); cognitive disorders such as dementia; psychotic disorders such as schizophrenia and mania; anxiety disorders; personality disorders such as ADHD; eating disorders such as anorexia nervosa and bulimia nervosa; chemical dependencies resulting from addictions to drugs or substances of abuse such as addictions to nicotine, alcohol, cocaine, heroin, phenobarbital and benzodiazepines; withdrawal syndromes; endocrine disorders such as hyperprolactinaemia; impulse disorders such as trichotillomania and kleptomania; tic disorders such as Tourette's syndrome; gastrointestinal tract disorders such as irritable bowel syndrome (IBS), ileus, gastroparesis, peptic ulcer, gastroesophageal reflux disease (GORD, or its synonym GERD), flatulence and other functional bowel disorders such as dyspepsia (e.g., non-ulcerative dyspepsia (NUD)) and non-cardiac chest pain (NCCP); vascular disorders including vasospasms such as in the cerebral vasculature; and miscellaneous other disorders, including Parkinson's disease, shock and hypertension, sexual dysfunction, pre-menstrual syndrome and fibromyalgia syndrome.

As discussed above, (−)-E-10-OH-NT also inhibits NE and 5HT transporters and the uptake of NE and/or 5HT. Accordingly, in yet another aspect, the present disclosure provides methods of inhibiting uptake of NE and/or 5HT. The methods generally comprise contacting a NE and/or 5HT transporter with an amount of (−)-E-10-OH-NT effective to inhibit uptake of NE and/or 5HT. In some embodiments, the method is carried out in the absence of AT and NT. In some embodiments, the NE and/or 5HT transporter is contacted with an E-10-OH-NT composition as described herein. In some embodiments, the E-10-OH-NT composition comprises E-10-OH-NT that is enriched in the (−) enantiomer. In some embodiments, the E-10-OH-NT composition comprises substantially enantiomerically pure (−)-E-10-OH-NT. In some embodiments, the E-10-OH-NT composition comprises enantiomerically pure (−)-E-10-OH-NT.

The methods can be practiced in vitro with isolated transporters or cells that express one or both transporters, or in vivo as a therapeutic approach towards the treatment of diseases or disorders that are, at least in part, mediated by dysregulated uptake or reuptake of NE and/or 5HT. Specific examples of diseases or disorders that are, at least in part, mediated by dysregulated uptake or reuptake of NE and/or 5HT include, but are not limited to, those listed above.

Historically, antidepressants including those that inhibit reuptake of NE (NRIs) and/or 5HT (SRIs) have been used as a first-line therapy for treating both acute and chronic pain that is either nociceptive or non-nociceptive in origin, for example, neuropathy, post-herpetic neuralgia (PHN), pain associated with fibromyalgia, pain associated with irritable bowel syndrome and interstitial cystitis (Sindrup & Jensen, 1999; Collins et al., 2000; Crowell et al., 2004). A recent study systematically evaluated the relative activity at the NE and/or 5HT transporter required for maximal efficacy in rodent models of pain (Leventhal et al., 2007). The effects observed replicate those observed clinically for treating neuropathic pain conditions. Namely, compounds with greater affinity for the NE transporter are more effective at treating pain and compounds with greater affinity for the 5HT transporter have limited efficacy (see, e.g., Max et al., 1992; Collins et al., 2000). Indeed, in a double-blind, placebo-controlled head-to-head study comparing the tetracyclic NRI maprotiline and the SRI paroxetine, reduction in pain intensity was significantly greater for study completers randomized to maprotiline (45%) as compared to paroxetine (26%) or placebo (27%) (Atkinson et al., 1999). Recently, duloxetine, a dual SRI and NRI with potency at both 5HT and NE transporters, was the first reuptake inhibitor approved for the treatment of diabetic neuropathy (Bymaster et al., 2005; Goldstein et al., 2005).

The NRI activity demonstrated herein for (−)-E-10-OH-NT makes this compound ideally suited for the treatment of many different types of pain syndromes. Indeed, in experiments carried out by the applicants and reported herein, (−)-E-10-OH-NT exhibited robust therapeutic efficacy in rodent models of both nociceptive inflammatory pain (see Examples 6, 15 and 16) and non-nociceptive neuropathic pain (see Examples 5 and 17-20) pain. In both models, the efficacy observed for (−)-E-10-OH-NT was equivalent to that of AT.

Accordingly, in yet another aspect, the present disclosure provides methods of treating pain in mammals, including humans. The methods generally comprise administering to a mammal suffering from pain, including a human, an amount of an E-10-OH-NT composition described herein effective to treat the pain. In some embodiments, the E-10-OH-NT composition comprises E-10-OH-NT that is enantiomerically enriched in (−) enantiomer. In some embodiments the E-10-OH-NT composition comprises substantially enantiomerically pure (−)-E-10-OH-NT. In some embodiments, the composition comprises enantiomerically pure (−)-E-10-OH-NT.

The methods can be used to treat numerous different types of pain syndromes, including acute or chronic pain that is either nociceptive in origin (for example somatic or visceral) or non-nociceptive in origin (for example neuropathic or sympathetic). In some embodiments, the pain is nociceptive pain including, but not limited to, inflammatory pain such as that associated with IBS or rheumatoid arthritis, pain associated with cancer, and pain associated with osteoarthritis. In some embodiments the pain is non-nociceptive pain including, but not limited to, neuropathic pain such as post-herpetic neuralgia (PHN), trigeminal neuralgia, focal peripheral nerve injury, anesthesia clolorosa, central pain (for example, post-stroke pain, pain due to spinal cord injury or pain associated with multiple sclerosis), and peripheral neuropathy (for example, diabetic neuropathy, inherited neuropathy or other acquired neuropathies).

The E-10-OH-NT composition can be administered alone, or it can be administered in combination with, or adjunctively to, one or more other drugs useful for treating pain and/or other indications. Specific non-limiting examples of drugs that can be used in combination with, or adjunctively to, the E-10-OH-NT compositions in a pain treatment or management regimen are provided in a later section.

The E-10-OH-NT compositions described herein are expected to provide significant advantages over drugs currently used to treat and/or manage pain, and in particular neuropathic pain syndromes. Most common tricyclic antidepressants used for treating pain antagonize the H₁ histaminergic receptor, and are therefore associated with significant weight gain and sedative effects. As mentioned above, (−)-E-10-OH-NT antagonizes this receptor to a significantly lesser extent, and has proven to not induce sedation in a rodent model of sedation at doses of (−)-E-10-OH-NT demonstrated herein to be effective in vivo in treating both nociceptive inflammatory and non-nociceptive neuropathic pain. Thus, the E-10-OH-NT compositions described herein provide a means of treating or managing pain while minimizing weight gain and sedation.

The E-10-OH-NT compositions described herein are expected to provide additional significant advantages when used to treat pain, or any of the other many diseases and indications described herein. For example, AT and NT are known inhibitors of cytochrome P₄₅₀ isoenzymes CYP2D6 and CYP2C19, and as a consequence are contraindicated for use with several important drugs metabolized by these enzymes. Exemplary drugs that are known to be metabolized at least in part by CYP2D6, and may therefore be contraindicated for use with AT and NT, include S-metoprolol, propafenone, timolol, clomipramine, desipramine, imipramine, paroxetine, haloperidol, risperidone, thioridazine, aripiprazole, codeine, dextromethorphan, duloxetine, flecainide, mexiletine, ondansetron, tamoxifen, tramadol and venlafaxine. Exemplary drugs that are known to be metabolized at least in part by CYP2C19, and may therefore be contraindicated for use with AT and NT, include omeprazole, lansoprazole, pantoprazole, rabeprazole, diazepam, phenyloin, phenobarbitone, clomipramine, cyclophosphamide and progesterone. Data obtained by the present applicants and reported herein (discussed in more detail in the Examples section) demonstrate that (−)-E-10-OH-NT is a less potent inhibitor of both CYP2C19 and CYP2D6 than AT and NT. Based in part on this surprising discovery, it is believed that the E-10-OH-NT compositions described herein would engender fewer undesirable clinical consequences than AT and/or NT therapy, especially when used in combination with, or adjunctively to, drugs that are metabolized at least in part by these cytochrome P450 isoenzymes.

It has also been surprisingly discovered by the applicants that (−)-E-10-OH-NT has a significantly lower affinity for muscarinic receptors than AT and NT, exhibits a lower inhibition of human Ether-a-Go-go related gene (hERG) potassium channels than AT and NT, and is less antagonistic at adrenergic receptors, including both α₁, and α₂ adrenergic receptors, than AT and NT. All of these properties are expected to result in improved clinical benefits as compared to AT and NT therapy. For example, inhibition of muscarinic receptors has been linked to dry mouth, constipation and blurred vision. Agonists of the α₂ adrenergic receptors have been reported to produce analgesia (Ongioco et al., 2000; Asano et al., 2000; Hall et al., 2001), while antagonists inhibit these analgesic effects (Kalso et al., 1991; Millan & Colpaert 1991; Petrovaara et al., 1990). While not intending to be bound by any theory of operation, it is expected that patients treated with the E-10-OH-NT compositions described herein will exhibit fewer undesirable side effects than patients treated with AT and/or NT. Specifically, it is expected that patients treated with the E-10-OH-NT compositions described herein will experience, in addition to the reduced drug-drug interactions and sedation and appetite effects discussed above, lower levels of cardiotoxicity and constipation than patients treated with AT and NT. Indeed, at equivalent oral doses, (−)-E-10-OH-NT produced much less constipation than AT and NT in a rodent model of gastrointestinal motility.

Unlike AT and NT, (−)-E-10-OH-NT does not antagonize α₂ adrenergic receptors. Therefore, in theory, the E-10-OH-NT compositions described herein are also expected to be superior to AT and NT in the treatment of pain. Indeed, as illustrated in FIGS. 3 & 4, (−)-E-10-OH-NT proved superior to AT in rodent models of both nociceptive and non-nociceptive pain.

Although the synthesis of (±)-E-10-OH-NT has been reported in the literature (Remy et al., 1973), to date, the chiral synthesis of the individual (+) and (−) enantiomers has never been reported. Thus, in still another aspect, the present disclosure provides chiral-specific methods of synthesizing (+) and (−) enantiomers of E-10-OH-NT, intermediates useful for the methods, and chiral specific methods of synthesizing the intermediates.

In some embodiments, intermediates useful for the chiral-specific synthesis of (+)-E-10-OH-NT and (−)-E-10-OH-NT are (+) and (−) enantiomers of E-5-(γ-bromopropylidene)-10,11-dihydro-10-hydroxy-5H-dibenzo[a,d]-cycloheptene, respectively, illustrated as compounds (+)-7 and (−)-7, respectively, in FIG. 1A. The absolute configuration of the chiral 10-carbon of the (+) and (−) enantiomers of compounds 7 have not been established directly. Accordingly, the bond connecting the hydroxyl group to this chiral carbon atom (chirality is indicated with an asterisk) is illustrated with unspecified stereochemistry. However, the chirality of compounds 7 may be inferred from the absolute stereochemistry of (−)-E-10-OH NT (depicted in FIG. 6), which has been determined by X-ray crystallographic analysis of the malate salt of (−)-E-10-OH NT.

Methods of synthesizing chiral intermediates (+)-7 and (−)-7 generally comprise reducing E-5-(γ-bromopropylidene)-10,11-dihydro-10-oxo-5H-dibenzo[a,d]-cycloheptene (illustrated as compound 6 in FIG. 1A) in the presence of a chiral-specific oxazaborolidine catalyst. Non-limiting exemplary embodiments of suitable chiral-specific oxazaborolidine catalysts are illustrated in FIG. 1B. In some embodiments, the chiral-specific oxazaborolidine catalyst is (7αS)-3-methyl-1,1-diphenylperhydro-3-bora-2-oxapyrrolizine (“7αS-CBS”; compound S-10 in FIG. 1A), yielding (−)-7. In some embodiments, the chiral-specific oxazaborolidine catalyst is (7αR)-3-methyl-1,1-diphenylperhydro-3-bora-2-oxapyrrolizine (“7αR-CBS”; compound R-10 in FIG. 1B), yielding (+)-7.

(+)-E-10-OH-NT and (−)-E-10-OH-NT can be synthesized from intermediates (+)-7 and (−)-7, respectively, by reacting the intermediates with methylamine. A specific embodiment of the synthesis utilizing CBS as the chiral-specific catalyst is illustrated in FIG. 1A. Utilizing this scheme under the reaction conditions provided in the Examples section, (+)-E-10-OH-NT and (−)-E-10-OH-NT can be readily synthesized in high yield with an enantiomeric purity of greater than 99%.

### 7. BRIEF DESCRIPTION OF THE FIGURES

FIG. 1A provides an exemplary scheme that can be used to synthesize enantiomerically pure (+)-E-10-OH-NT and (−)-E-10-OH-NT;

FIG. 1B provides exemplary embodiments of chiral-specific oxazaborolidine catalysts that can be used in the synthetic scheme illustrated in FIG. 1A;

FIG. 2 provides a graph demonstrating the non-sedative effects of (−)-E-10-OH-NT as compared to AT and NT in the rat rotarod model of sedation;

FIG. 3 provides a graph demonstrating the superior antihyperalgesic efficacy of (−)-E-10-OH-NT as compared to AT in a rodent model of nociceptive inflammatory pain;

FIG. 4 provides a graph demonstrating the superior antiallodynic efficacy of (−)-E-10-OH-NT as compared to AT in a rodent model of non-nociceptive neuropathic pain;

FIG. 5 provides a graph demonstrating the superior properties of (−)-E-10-OH-NT on gastrointestinal tract mobility as compared to both AT and NT in a rodent model of GIT;

FIG. 6 depicts the absolute configuration of (−)-E-10-OH-NT established by X-ray crystallographic analysis;

FIG. 7 provides a graph demonstrating the superior antiallodynic efficacy of (−)-E-10-OH-NT as compared to (+)-E-10-OH-NT, racemic (±)-E-10-OH-NT and AT in a rodent model of non-nociceptive neuropathic pain;

FIG. 8 provides a graph demonstrating the antiallodynic efficacy of orally-administered (−)-E-10-OH-NT as compared to that of amitriptyline in a rodent model of non-nociceptive neuropathic pain;

FIG. 9 provides a curve depicting the FCA and rotarod dose-response relationships for amitriptyline in a rodent model of nociceptive inflammatory pain;

FIG. 10 provides a curve depicting the FCA and rotarod dose-response relationships for (−)-E-10-OH-NT in a rodent model of nociceptive inflammatory pain;

FIG. 11 provides a graph demonstrating the dose-related antihyperalgesic activity of (−)-E-10-OH-NT in a rodent model of nociceptive inflammatory pain;

FIG. 12 provides a graph demonstrating the absence of substantial antihyperalgesic activity exhibited by (+)-E-10-OH-NT in a rodent model of nociceptive inflammatory pain;

FIG. 13 provides a graph demonstrating the differential antihyperalgesic activity of the enantiomers of E-10-OH-NT in a rodent model of nociceptive inflammatory pain;

FIG. 14 provides a graph comparing the antihyperalgesic activity of the enantiomers of E-10-OH-NT in a rodent model of nociceptive inflammatory pain;

FIG. 15A provides a graph demonstrating the activity of amitriptyline (AT) in the rat Forced Swim Test model of depression;

FIG. 15B provides a graph demonstrating the activity of (−)-E-10-OH-NT in the rat Forced Swim Test model of depression; and

FIG. 15C provides a graph demonstrating that, at the dosages tested, (+)-E-10-OH-NT did not display statistically significant activity in the rat Forced Swim Test model of depression.

### 8. DETAILED DESCRIPTION

8.1 E-10-OH-NT Compounds and Compositions

The present disclosure concerns compositions comprising the E-geometrical isomers of the common major metabolite of the well-known tricyclic antidepressants amitriptyline (AT) and nortriptyline (NT): (S/R)-5-[3-Methylamino-prop-(E)-ylidene]-10,11-dihydro-5H-dibenzo[a,d]cyclohepten-10-ol (referred to herein as “E-10-OH-NT”), illustrated below:

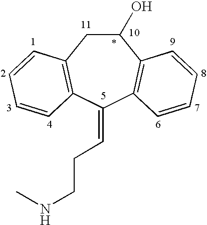

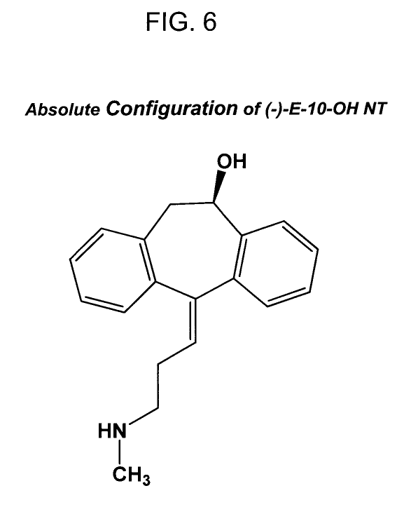

Owing to the chirality at the carbon at the 10-position (indicated with an asterisk), the E-geometrical isomer comprises two enantiomers: (+)-E-10-OH-NT and (−)-E-10-OH-NT. The absolute configurations about the chiral 10-carbon of the (−) isomer, established by x-ray crystallographic analysis, is depicted in FIG. 6. Therefore the chirality of the (−) and (+) isomers is as depicted below:

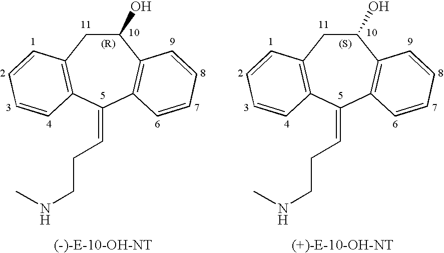

In the various compositions described herein, the E-10-OH-NT compound can be present as a non-racemic mixture enriched in the (−) enantiomer, as the substantially enantiomerically pure (−) enantiomer or as the enantiomerically pure (−) enantiomer. In a specific embodiment, the E-10-OH-NT compositions described herein comprise E-10-OH-NT that is substantially enantiomerically pure (−)-E-10-OH-NT. In another specific embodiment, the E-10-OH-NT compositions described herein comprise E-10-OH-NT that is enantiomerically pure (−)-E-10-OH-NT.

As used herein, a composition is “enriched” in a particular enantiomer when that enantiomer is present in excess over the other enantiomer, i.e., when that enantiomer comprises more than 50% of the total E-10-OH-NT in the composition. A composition that is enriched in a particular enantiomer will typically comprise at least about 60%, 70%, 80%, 90%, or even more, of the specific enantiomer. The amount of enrichment of a particular enantiomer can be confirmed using conventional analytical methods routinely used by those skilled in the art, including NMR spectroscopy in the presence of chiral shift reagents, gas chromatographic analysis using chiral columns, and high pressure liquid chromatographic analysis using chiral columns.

In some embodiments, a single enantiomer will be substantially free of the corresponding enantiomer. By “substantially free of” is meant that the composition comprises less than about 10% of the specified undesired enantiomer as established using conventional analytical methods routinely used by those of skill in the art, such as the methods mentioned above. In some embodiments, the amount of undesired enantiomer may be less than 10%, for example, less than 9%, 8%, 7%, 6%, 5%, 4%, 3%, 2%, 1% or even less. Enantiomerically enriched compositions that contain at least about 95% of a specified enantiomer are referred to herein as “substantially enantiomerically pure.” Compositions that contain at least about 99% of a specified enantiomer are referred to herein as “enantiomerically pure.”

In some embodiments, the enantiomerically enriched E-10-OH-NT compositions described herein contain E-10-OH-NT that is approximately 60%, 70%, 80% or 90% pure in the (−)-E-10-OH-NT. Stated another way, the (−) enantiomer is present at an enantiomeric excess (ee) in the range of about 60, 70, 80 or 90% ee. In some specific embodiments, the substantially enantiomerically pure E-10-OH-NT compositions described herein contain E-10-OH-NT that is approximately 95-98% pure in the (−) enantiomer; stated another way, the (−)-E-10-OH-NT is present in an enantiomeric excess in the range of about 95-98% ee). In some specific embodiments, the enantiomerically pure E-10-OH-NT compositions described herein contain E-10-OH-NT that is approximately 99.0 to 100% pure in the (−) enantiomer; stated another way, the (−)-E-10-OH-NT is present in an enantiomer excess in the range of about 99.0 to 100% ee). Specific, non-limiting exemplary embodiments include E-10-OH-NT compositions in which the E-10-OH-NT is approximately 99.0, 99.1, 99.2, 99.3, 99.4, 99.5, 99.6, 99.7, 99.8, 99.9 or 100% pure in the (−) enantiomer.

In vivo, the hydroxymetabolites of tricyclic antidepressant drugs are formed by the polymorphic cytochrome P450 enzyme CYP2D6. NT is thought to be hydroxylated by this enzyme in a highly stereospecific way to the (−) enantiomer of E-10-OH-NT (see, e.g., Nordin & Bertilsson, 1995). Thus, in theory, (−)-E-10-OH-NT can be isolated from biological sources at an enantiomeric purity of 100%. In contrast, skilled artisans will appreciate that compositions of (−)-E-10-OH-NT produced by synthetic means typically will not achieve enantiomeric purities of 100%. For example, in the synthetic methods described in the Examples section, enantiomeric purities in the range of about 90 to 99.5% ee of the (−) enantiomer are typically achieved.

Unless specifically noted otherwise, the enantiomerically pure E-10-OH-NT compositions described herein are intended to include enantiomerically pure (−)-E-10-OH-NT of both biological and synthetic origins. Thus, the enantiomerically pure E-10-OH-NT compositions described herein can contain from about 99% up to 100% enantiomerically pure (−)-E-10-OH-NT.

Embodiments of enantiomerically pure E-10-OH-NT compositions that are of biological origin (i.e., isolated from biological sources) and that therefore may contain 100% (−)-E-10-OH-NT, are referred to herein as “biologically derived enantiomerically pure (−)-E-10-OH-NT compositions.” Likewise, such E-10-OH-NT is referred to herein as “biologically derived enantiomerically pure (−)-E-10-OH-NT.” Embodiments of enantiomerically pure E-10-OH-NT compositions that are of synthetic origin, including compositions prepared ex vivo with the aid of chiral specific biocatalysts such as CYP2D6, and that therefore contain at least about 99%, but typically less than 100% (−)-E-10-OH-NT, are referred to herein as “synthetically derived enantiomerically pure (−)-E-10-OH-NT compositions.” Likewise, such E-10-OH-NT is referred to herein as “synthetically derived enantiomerically pure (−)-E-10-OH-NT.” Synthetically derived enantiomerically pure (−)-E-10-OH-NT will typically contain the (−) enantiomer in an enantiomeric excess in the range of about 99 to 99.9% ee.

Depending upon the intended use, the E-10-OH-NT can be present in the composition as the free base, or in the form of a salt, for example, an acid additional salt. In some embodiments, the E-10-OH-NT is present in the composition in the form of a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-tuluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the E-10-OH-NT is present in the composition as an organic acid addition salt, for example, an acid addition salt formed with an organic acid selected from D-malic acid, L-malic acid and succinic acid.

It is well known in the art that different salt forms of compounds may exhibit different properties, such as, for example, different toxicities, solubilities, stabilities, hygroscopicities, etc. It has been discovered that (−)-E-10-OH-NT acid addition salts formed with succinic acid (succinate salts) have superior solubility in saline (18 mg/ml) than (−)-E-10-OH-NT salts formed with maleic acid (4.4 mg/ml), and are crystalline in nature and non-hygroscopic. Accordingly, while the use of the maleic acid salt form of racemic (±)-E-10-OH-NT has been previously used to dose healthy volunteers (Bertilsson et al., 1986), it is expected that the succinate salts of (−)-E-10-OH-NT may have superior properties. For example, maleic acid has been previously associated with acute tubular necrosis toxicity (Everett et al., 1993). It is anticipated that the succinate salt will have superior ADME and toxicological safety properties than the maleate salt. Accordingly, in some embodiments, the (−)-E-10-OH-NT constituting the composition is a succinate salt.

The E-10-OH-NT, whether in the form of a free base or salt, can be present in the composition in anhydrous form, or it can be in the form of a solvate and/or hydrate. The degree and identity of solvating solvent will depend, in part, in the conditions used to synthesize and store the E-10-OH-NT compound. As used herein, unless specifically indicated otherwise, the expressions “E-10-OH-NT,” (±)-E-10-OH-NT, (+)-E-10-OH-NT and (−)-E-10-OH-NT are intended to include all salt and/or solvate and/or hydrate forms of the compounds.

8.2 Methods of Synthesis

The (−)-E-10-OH-NT compound can be synthesized or prepared using methods described in the literature, for example, racemic (+)-E-10-OH-NT can be synthesized as described in Bertrand et al., 1994 and Lassen et al., 1983, the disclosures of which are incorporated herein by reference. Enantiomerically enriched compositions of (−)-E-10-OH-NT can be prepared from such racemic mixtures by isolating the desired enantiomers using standard methods of chiral separation (see, e.g., Chiral Separation Techniques: A Practical Approach 2001).

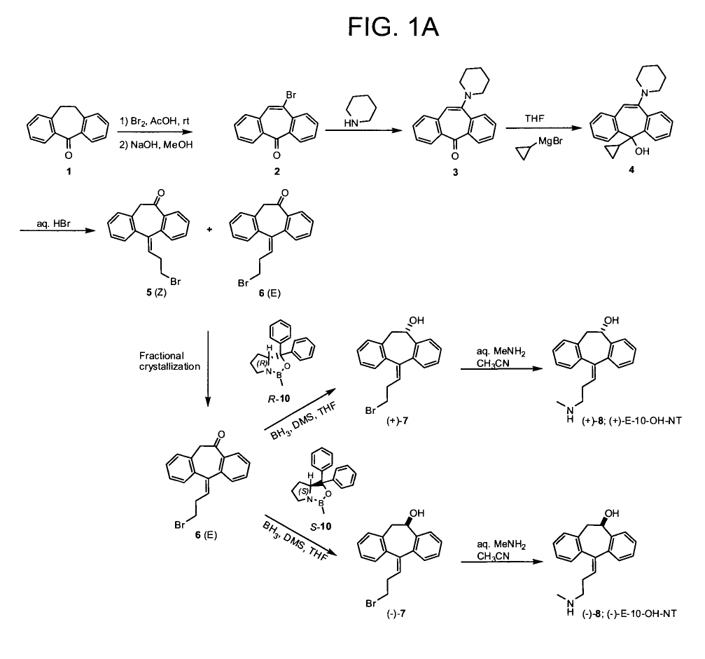

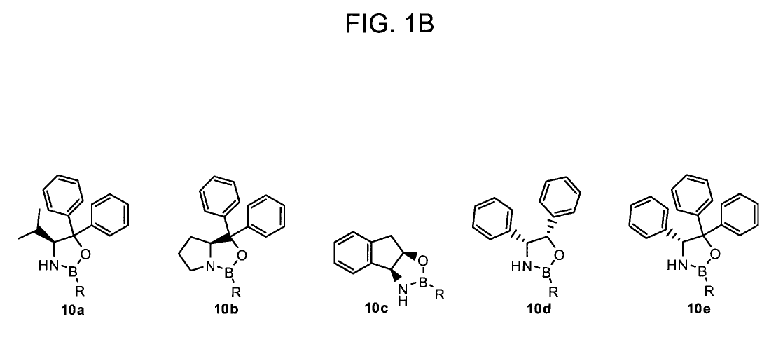

Another aspect of the disclosure provides means of synthesizing enantiomerically pure isomers of E-10-OH-NT using chiral specific synthesis methods. An exemplary embodiment of methods useful for synthesizing enantiomerically pure (+)-E-10-OH-NT and (−)-E-10-OH-NT is illustrated in FIG. 1A. According to FIG. 1A, the E-isomers of 5-(γ-bromopropylidene)-10,11-dihydro-10-oxo-5H-dibenzo[a,b]-cycloheptene (compound 6) is reduced in the presence of a chiral-specific oxazaborolidine catalyst (illustrated as compounds S-10 and R-10). As illustrated, the S-isomer of the catalyst (S-10) yields the (−) enantiomer of 5-(γ-bromopropylidene)-10,11-dihydro-10-oxo-5H-dibenzo[a,b]-cycloheptene (compound (−)-7), and the R-isomer (R-10) yields the (+)-enantiomer, (+)-7. These intermediates can then be converted to their respective (+)-E-10-OH-NT and (−)-E-10-OH-NT products by reaction with methylamine.

The compound 6 starting material can be synthesized using methods described in the literature (see, e.g., Bertrand et al., 1994; Lassen et al., 1983, the disclosures of which are incorporated by reference). Alternatively, they can be synthesized as illustrated in FIG. 1A. Specific conditions for each illustrated step are provided in the Examples section.|

In FIG. 1A, a specific chiral-specific oxazaborolidine catalyst is illustrated. Other chiral-specific oxazaborolidine catalysts could also be used. Specific representation examples are illustrated in FIG. 1B (in FIG. 1B, R is hydrogen or methyl) and described in Itsuno et al., 1983 (catalyst 10a), Corey et al., 1987 (catalyst 10b), Hong et al., 1994 (catalyst 10c), Quallich et al., 1993 (catalyst 10d) and Berenguer et al., 1994 (catalyst 10e). The asymmetric induction achieved by the CBS reducing system is highly influenced by the variant group attached to the boron atom. This group can be tuned to fit a particular substrate, although most commonly B-hydrogen or B-methyl is used. The group attached to the boron atom directs the ketones to bind in such a way that there is minimal interaction between the B-R group and the small substituent of the ketone (for details regarding mechanistic studies, see Kim et al., 2006). General conditions under which the reduction can be carried out are described in the aforecited references and also in Corey et al., 1991; Corey et al., 1992; Corey et al., 1998; Paterson et al., 2005; Zartman et al., 2005; Tagat et al., 2004; Duquette et al., 2003; Yanagi et al., 2003; and Coe et al., 2003, the disclosures of which are incorporated herein by reference. The reaction can be carried out using the specific catalyst illustrated in FIG. 1A in high yield and with an enantiomeric purity in excess of 99% when performed at −20° C. in THF in the presence of borane-dimethyl sulfide complex, as described in the Examples section.

8.3 Activities and Uses of the Compounds and Compositions

As described in more detail in Examples 4 and 14, like AT and NT, (−)-E-10-OH-NT has a strong affinity for the norepinephrine (“NE”) transporter, and is a potent inhibitors of NE uptake. (−)-E-10-OH-NT also binds the serotonin transporter and inhibits uptake of 5HT, albeit with less potency than that observed for NE. The use of compounds that inhibit reuptake of NE and/or 5HT to treat a variety of diseases and disorders is well documented. For example, AT, NT, desipramine, duloxetine, venlafaxine, citalopram, and simbalta are approved for the treatment of depression and have additional off label uses; and paroxetine and sertraline are approved for treatment of major depressive disorder, OCD, panic disorder, post traumatic stress disorder, premenstrual dysphoric disorder and social anxiety disorder, and also have additional off label uses.

The ability of racemic E-10-OH-NT to cross the blood-brain barrier has been established in the literature (see, e.g., Nordin et al., 1987b). Accordingly, the E-10-OH-NT compositions described herein are expected to be useful to treat any disease and/or indication mediated, at least in part, by dysregulated NE reuptake. In some specific embodiments, it is expected that the E-10-OH-NT compositions described herein will be useful to treat many different diseases that respond to treatment with other NRI and SNRI agents, including, by way of example and not limitation, AT, NT, atomoxetine, reboxetine, and maprotiline. Diseases and disorders known to be mediated, at least in part, by dysregulated NE reuptake, and that are known to respond to treatment with NRI and SNRI compounds include, but are not limited to, urinary disorders, including urinary incontinence; mood disorders such as depression and seasonal affective disorder (SAD); cognitive disorders such as dementia; psychotic disorders such as schizophrenia and mania; anxiety disorders; personality disorders such as ADHD; eating disorders such as anorexia nervosa and bulimia nervosa; chemical dependencies resulting from addictions to drugs or substances of abuse such as addictions to nicotine, alcohol, cocaine, heroin, phenobarbital and benzodiazepines; withdrawal syndromes; endocrine disorders such as hyperprolactinaemia; impulse disorders such as trichotillomania and kleptomania; tic disorders such as Tourette's syndrome; gastrointestinal tract disorders such as irritable bowel syndrome (IBS), ileus, gastroparesis, peptic ulcer, gastroesophageal reflux disease (GORD, or its synonym GERD), flatulence and other functional bowel disorders such as dyspepsia (e.g., non-ulcerative dyspepsia (NUD)) and non-cardiac chest pain (NCCP); vascular disorders including vasospasms such as in the cerebral vasculature; and miscellaneous other disorders, including Parkinson's disease, shock and hypertension, sexual dysfunction, pre-menstrual syndrome and fibromyalgia syndrome. Indeed, as demonstrated in Example 21, (−)-E-10-OH-NT exhibits efficacy in rodent models of depression.

Pain is also thought to be mediated, at least in part, by dysregulated NE and/or 5-HT reuptake. Indeed, many NRI and/or SRI compounds are also used off-label to treat pain. Pain is generally understood to refer to the perception or condition of unpleasant sensory or emotional experience, which may or may not be associated with actual damage to tissues. It is generally understood to include two broad categories: acute and chronic (See, e.g., Buschmann et al., 2002; Jain, 2000) that is either of nociceptive (for example somatic or visceral) or non-nociceptive (for example neuropathic or sympathetic) in origin. Acute pain generally includes nociceptive pain arising from strains/sprains, burns, myocardial infarction, acute pancreatitis, surgery, trauma and cancer. Chronic pain generally includes nociceptive pain including, but not limited to, inflammatory pain such as that associated with IBS or rheumatoid arthritis, pain associated with cancer and pain associated with osteoarthritis; and non-nociceptive pain including, but not limited to, neuropathic pain such as post-herpetic neuralgia (PHN), trigeminal neuralgia, focal peripheral nerve injury, anesthesia clolorosa, central pain (for example, post-stroke pain, pain due to spinal cord injury or pain associated with multiple sclerosis), and peripheral neuropathy (for example, diabetic neuropathy, inherited neuropathy or other acquired neuropathies).

Animal data presented in the Examples section confirm that (−)-E-10-OH-NT is effective at treating both nociceptive inflammatory and non-nociceptive neuropathic pain. Accordingly, in some embodiments, the E-10-OH-NT compositions described herein are used to treat pain, including the various types pain discussed above. As noted above, in certain embodiments, such compositions comprise substantially enantiomerically pure (−)-E-10-OH-NT. In some embodiments, such compositions comprise enantiomerically pure (−)-E-10-OH-NT.

As demonstrated herein, racemic (±)-E-10-OH-NT, (+)-E-10-OH-NT and (−)-E-10-OH-NT exhibited similar affinities for the norepinephrine and serotonin transporters (Example 14), and the (−) and (+) isomers of E-10-OH-NT exhibited similar in vitro activity towards six different types of receptors and transporters, i.e., the norepinephrine transporter (Examples 4 and 14), serotonin transporter (Examples 4 and 14), dopamine transporter (Example 4), histaminergic receptor (Example 7), α adrenergic receptor (Example 9), and muscarinic receptors (Example 12), and also toward Cytochrome P450 functions (Example 10) and hERG ion channel (Example 11). In view of this data, particularly when taken collectively, it would be expected that the racemate and (+) and (−) enantiomers would have similar efficacies in vivo. Notwithstanding that expectation, the data presented herein unexpectedly demonstrate that the (−) enantiomer of E-10-OH-NT is therapeutically more effective than both the racemate and the (+) enantiomer in rodent models of pain, and more effective than the (+) enantiomer in a rodent model of depression. More specifically, at the dosages tested: (1) (−)-E-10-OH-NT was more effective than both racemic (±)-E-10-OH-NT and (+)-E-10-OH-NT in the rodent L5-Single Nerve Ligation model of non-nociceptive neuropathic pain (Example 15); (2) (−)-E-10-OH-NT was more effective than (+)-E-10-OH-NT in the FCA-induced hyperalgesia assay of nociceptive pain (Examples 19 and 20); and (3) (−)-E-10-OH-NT was more effective than (+)-E-10-OH-NT in the rat Forced Swim Test model of depression (Example 21). These discoveries could not have been predicted based on the in vitro data generated with racemic E-10-OH-NT and its (+) and (−) enantiomers. Indeed, based on the lack of efficacy observed in with (±)-E-10-OH-NT in the rat L5 SNL model of pain, one would have had no reason to test the isolated enantiomers in that, or any other, in vivo animal model of disease.

When used to treat various diseases or indications, the E-10-OH-NT composition will generally be administered in amounts effective to treat the particular disease or indication. As will be recognized by skilled artisans, what is understood to be “therapeutically effective” and providing therapeutic benefit oftentimes depends upon the specific disease or indication. Skilled artisans will be able to ascertain a therapeutically effective amount based upon long established criteria for the particular indication.

In general, a “therapeutically effective” amount of a composition is an amount that eradicates or ameliorates the underlying disease or indication being treated and/or that eradicates or ameliorates one or more of the symptoms associated with the underlying disorder such that the patient reports an improvement in feeling or condition, not withstanding that the patient may still be afflicted with the underlying disease or indication. Therapeutic benefits also includes halting or slowing the progression of the disease or indication, regardless of whether improvement is realized.

In the context of depression, a therapeutically effective amount is an amount of composition that eradicates or ameliorates the depression or the symptoms thereof, including, but not limited to, changes in mood, feeling of intense sadness, despair, mental slowing, loss of concentration, pessimistic worry, agitation, self-deprecation, insomnia, anorexia, weight loss, decreased energy and libido, and hormonal circadian rhythms.

In the context of anxiety disorder, a therapeutically effective amount is an amount of composition that eradicates or ameliorates the anxiety disorder or one of the symptoms thereof including, but are not limited to, a fear of losing control of one's own actions, a sense of terror arising from no apparent reason, a dread of catastrophe, uneasiness, nervousness, nagging uncertainty about future events, headaches, fatigue, and sub-acute autonomic symptoms.

In the context of pain, a therapeutically effective amount is an amount of composition that eradicates or ameliorates the pain or the symptoms thereof including, but not limited to, shooting sensations, burning sensations, electrical sensations, aching, discomfort, soreness, tightness, stiffness, sleeplessness, numbness, and weakness.

8.4 Combination Therapies

AT and NT have been used in combination with other agents to treat various diseases and disorders. For example, AT has been used in combination with chlordiazepoxide to treat anxiety disorder and major depressive disorder, and has been used in combination with perphenazine to treat anxiety disorder, schizophrenia and major depressive disorder. Additionally, NT has been used in combination with budenoside to treat asthma. Given that AT, NT and the E-10-OH-NT compositions described herein inhibit uptake of NE and 5HT, it is expected that the E-10-OH-NT compositions described herein will also be useful in combination therapies.

When used in combination therapy, the E-10-OH-NT compositions described herein may be used in combination with or as an adjunct to other agents. When the E-10-OH-NT compositions described herein are used in combination with other agents, the two agents may be administered in a single pharmaceutical compositor or they may be administered in separate pharmaceutical compositions. The two components may be administered by the same route of administration or by a different route of administration. The two components also may be administered simultaneously with each other or sequentially. Thus each component of the combination therapy may be administered separately but sufficiently closely in time to the administration of the other component as to provide the desired effect.

While combination therapy involving the E-10-OH-NT compositions described herein is useful in many contexts, the other agent used with the E-10-OH-NT compositions described herein will depend on the specific disease or indication being treated. The skilled artisan will be able to ascertain what other agent to use in combination with the E-10-OH-NT compositions described herein based upon long established criteria for the particular indication. While not intending to be bound by any theory of operation, the combination therapy may include the administration of the E-10-OH-NT compositions described herein with other agents known to inhibit the reuptake of NE and 5HT. Alternatively, the combination therapy may include the administration of the E-10-OH-NT compositions described herein with agents which do not inhibit the reuptake of NE and 5HT.

Accordingly, the E-10-OH-NT compositions described herein may be combined with other agents that inhibit reuptake of NE and/or 5HT, as well as, dual and triple monoamine uptake inhibits to treat depression. The E-10-OH-NT compositions described herein also can be combined with a selective serotonin reuptake inhibitor (SSRI) such as, but not limited to, fluxetine, paroxetine, fluvoxamine, citaprolam, and sertraline to treat depression. Combination therapy for the treatment of depression may also involve a monoamine oxidase inhibitor (MAOIs), such as, but not limited to, tranylcypromine, phenelzine, and isocarboxazid. Alternatively, the combination therapy may involve a heterocyclic antidepressant such as, but not limited to, amoxapine, maprotiline, and trazodone, or another antidepressants such as, but not limited to venlafaxine, nefazodone, and mirtazapine. Additionally, the combination therapy for the treatment of depression may involve the E-10-OH-NT compositions described herein and anti-anxiety agents, such as, but not limited to, chlordiazepoxide, or an anti-psychotic agents such as, but not limited to, perphenazine.

It is expected that the E-10-OH-NT compositions described herein, like AT and NT, will be useful in combination therapies for the treatment of anxiety disorder, schizophrenia and asthma. In the context of anxiety disorder, the E-10-OH-NT compositions described herein may be combined with anti-anxiety agents such as, but not limited to, chlordiazepoxide. In the context of schizophrenia, the E-10-OH-NT compositions described herein may be combined with an agent known to treat schizophrenia such as, but not limited to, perphenazine. In the context of asthma, the E-10-OH-NT compositions described herein may be combined with agents known to treat asthma such as corticosteroids, including, but not limited to, budenoside.

It is also expected that the E-10-OH-NT compositions described herein will be useful in combination therapy for the treatment of pain. Accordingly, the E-10-OH-NT compositions described herein can be combined with other analgesics, including but not limited to, cannabinoids and opioids. A number of cannabinoids are available that may be suitable for use in combination therapy. Accordingly, the combination therapy may involve a cannabinoid that is selected from Δ⁹-tetrahydrocannabinol and cannabidiol, and mixtures thereof.

Alternatively, the E-10-OH-NT compositions described herein may be used in combination with at least one opioid. A wide variety of opioids are available that may be suitable for use in combination therapy to treat pain. As such, the combination therapy may involve an opioid that is selected from, but not limited to, alfentanil, allylprodine, alphaprodine, anileridine, benzyl-morphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioaphetylbutyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, loperamide, meperidine (pethidine), meptazinol, metazocine, methadone, metopon, morphine, myrophine, nalbuphine, narceine, nicomorphine, norlevorphanol, normethadone, nalorphine, normorphine, norpinanone, opium, oxycodone, oxymorphone, papaveretum, pentazocine, phenadoxone, phenomorphan, phanazocine, phenoperidine, piminodine, piritramide, propheptazine, promedol, properidine, propiram, propoxyphene, sulfentanil, tilidine, tramadol, diastereoisomers thereof, pharmaceutically acceptable salts thereof, complexes thereof, and mixtures thereof. In some embodiments, the opioid is selected from morphine, codeine, oxycodone, hydrocodone, dihydrocodeine, propoxyphene, fentanyl, tramadol, and mixtures thereof.

The opioid component of the combination therapy may further include one or more other active ingredients that may be conventionally employed in analgesic and/or cough-cold-antitussive combination products. Such conventional ingredients include, for example, aspirin, acetaminophen, dextromethorphan, phenylpropanolamine, phenylephrine, chlorpheniramine, caffeine, and/or guaifenesin. Typical or conventional ingredients that may be included in the opioid component are described, for example, in the Physicians' Desk Reference, 1999, the disclosure of which is hereby incorporated herein by reference, in its entirety.

The opioid component may further include one or more compounds that may be designed to enhance the analgesic potency of the opioid and/or to reduce analgesic tolerance development. Such compounds include, for example, dextromethorphan or other NMDA antagonists (Mao et al., 1996), L-364,718 and other CCK antagonists (Dourish et al., 1988), NOS inhibitors (Bhargava et al., 1996), PKC inhibitors (Bilsky et al., 1996), and dynorphin antagonists or antisera (Nichols et al., 1997). The disclosures of each of the foregoing documents are hereby incorporated herein by reference, in their entireties.

Alternatively, the compounds described herein may be used with at least one non opioid analgesic, such as for example, diclofenac, a COX2 inhibitor, aspirin, acetaminophen, ibuprofen, naproxen, and the like, and mixtures thereof.

Additionally, in the context of treating pain, the combination therapy may involve an anti-inflammatory including, but not limited to, corticosteroids, aminoarylcarboxylic acid derivatives such as, but not limited to, etofenamate, meclofenamic acid, mefanamic acid, niflumic acid; arylacetic acid derivatives such as, but not limited to, acemetacin, amfenac cinmetacin, clopirac, diclofenac, fenclofenac, fenclorac, fenclozic acid, fentiazac, glucametacin, isozepac, lonazolac, metiazinic acid, oxametacine, proglumetacin, sulindac, tiaramide and tolmetin; arylbutyric acid derivatives such as, but not limited to, butibufen and fenbufen; arylcarboxylic acids such as, but not limited to, clidanac, ketorolac and tinoridine; arylpropionic acid derivatives such as, but not limited to, bucloxic acid, carprofen, fenoprofen, flunoxaprofen, ibuprofen, ibuproxam, oxaprozin, piketoprofen, pirprofen, pranoprofen, protizinic acid and tiaprofenic add; pyrazoles such as, but not limited to, mepirizole; pyrazolones such as, but not limited to, clofezone, feprazone, mofebutazone, oxyphenbutazone, phenylbutazone, phenyl pyrazolidininones, suxibuzone and thiazolinobutazone; salicylic acid derivatives such as, but not limited to, bromosaligenin, fendosal, glycol salicylate, mesalamine, 1-naphthyl salicylate, olsalazine and sulfasalazine; thiazinecarboxamides such as, but not limited to, droxicam, isoxicam and piroxicam; and other anti-inflammatory agents such as, but not limited to, e-acetamidocaproic acid, s-adenosylmethionine, 3-amino-4-hydroxybutyric acid, amixetrine, bendazac, bucolome, carbazones, difenpiramide, ditazol, guaiazulene, heterocyclic aminoalkyl esters of mycophenolic acid and derivatives, nabumetone, nimesulide, orgotein, oxaceprol, oxazole derivatives, paranyline, pifoxime, 2-substituted-4,6-di-tertiary-butyl-s-hydroxy-1,3-pyrimidines, proquazone and tenidap.

8.5 Formulations and Administration

The (−)-E-10-OH-NT compound or pharmaceutical salts thereof described herein may be combined with a pharmaceutical carrier selected on the basis of the chosen route of administration and standard pharmaceutical practice as described, for example, in Remington's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

The (−)-E-10-OH-NT compound and/or compositions disclosed herein, may be administered by any means that results in the contact of the active agent(s) with the desired site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

The (−)-E-10-OH-NT compound and/or compositions described herein may be administered to a mammalian host in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

The (−)-E-10-OH-NT compound and/or compositions may be formulated for oral administration, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained. Preferred compositions or preparations according to the present invention may be prepared so that an oral dosage unit form contains from about 0.1 to about 1000 mg of each active compound (and all combinations and subcombinations of ranges and specific concentrations therein).

The tablets, troches, pills, capsules and the like may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the dosage unit form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non toxic in the amounts employed.

The (−)-E-10-OH-NT compound and/or compositions may also be formulated for parental or intraperitoneal administration. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

Compositions suitable for administration by injection typically include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze drying technique that yields a powder of the active ingredient, plus any additional desired ingredient from the previously sterile filtered solution thereof.

8.6 Effective Dosages

The (−)-E-10-OH-NT compound and/or compositions will generally be administered in a therapeutically effective amount as described herein. The amount of compound or composition administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Dosage amounts will typically be in the range of from about 0.0001 or 0.001 or 0.01 mg/kg/day to about 0.1 or 1.0 or 2.0 or 2.5 or 5.0 or 10.0 or 20.0 or 25.0 or 50.0 or 75.0 or 100 mg/kg/day with an expected dose of about 5 mg/day to about 500 mg/day, but may be higher or lower, depending upon, among other factors, the particular disease or indication being treated the activity of the compound and/or composition, its bioavailability, the mode of administration and various factors discussed above. Dosage amount and interval may be adjusted individually to provide plasma levels of the compounds and/or compositions which are sufficient to maintain therapeutic or prophylactic effect. As non-limiting examples, the compounds and/or compositions may be administered once per day or multiple times per day, depending upon, among other things, the mode of administration, the specific indication being treated and the judgment of the prescribing physician. In cases of local administration or selective uptake, such as local topical administration, the effective local concentration of active compounds and/or compositions may not be related to plasma concentration. Skilled artisans will be able to optimize effective local dosages without undue experimentation.

Initial dosages of the (−)-E-10-OH-NT compound and/or compositions useful for the treatment of pain can be estimated from in vivo data, such as the animal models described in the Examples section. Additionally, initial dosages may be estimated from the data regarding the effective dosages of AT and NT for the treatment of the various disorders available in the art. See, e.g., Bryson & Wilde, 1996 (reporting a starting dose of 10 to 25 mg/day of AT for the treatment of chronic pain, with an increase of 10 to 25 mg/week to the maximum suggested dose, 75 mg/day, or tolerated dose) and the data available in the art regarding pharmacokinetic and pharmacodynamic properties of racemic (±)-E-10-OH-NT, and its separate enantiomers following the administration of racemic (±)-E-10-OH-NT, AT and/or NT (Dahl-Puustinen et al., 1989 (reporting mean AUC for the separated enantiomers following the administration of a single 75 mg oral dose of racemic (±)-10-OH-NT); Bertilsson et al., 1986 (reporting the pharmacokinetic properties of E-10-OH-NT following the administration of dosages ranging from 10 to 100 mg); Edelbroek et al., 1986 (reporting the steady state concentration of E-10-OH-NT following the administration of 75 mg of AT)).

Based on the animal models described in Examples 5, 6, and 15-21, it is believed that an effective dosage of (−)-E-10-OH-NT for the treatment of pain in humans may be obtained by administering a dose of (−)-E-10-OH-NT sufficient to achieve a plasma concentration similar to that achieved following the administration of 30 mg/kg, i.p. to rats. As such, in some embodiments the effective dose (−)-E-OH-NT for the treatment of pain is the dosage required to achieve the plasma concentration achieved when (−)-E-10-OH-NT (30 mg/kg, i.p.) is administered to rats.

Dosage amounts of (−)-E-OH-NT for treating pain will typically be in the range of from about 0.0001 or 0.001 or 0.01 mg/kg/day to about 0.1 or 1.0 or 2.0 or 2.5 or 5.0 or 10.0 or 20.0 or 25.0 or 50.0 or 75.0 or 100 mg/kg/day with an expected dose of about 5 mg/day to about 500 mg/day, but may be higher or lower. It is believed that oral doses of (−)-E-10-OH-NT of between about 10 mg/day to about 20 or 25 or 30 or 35 or 40 or 45 or 50 or 60 or 70 or 80 or 90 or 95 or 100 or 150 or 200 or 250 or 300 mg/day will be effective in treating pain. Accordingly, some embodiments involve the administration of an oral dosage of (−)-E-10-OH-NT between about 10 mg/day to about 100 mg/day.

Based on the Forced Swim animal model test described in Example 21, it is believed that an effective dosage of (−)-E-10-OH-NT for the treatment of depression in humans may be obtained by administering a dose of (−)-E-10-OH-NT sufficient to achieve a plasma concentration similar to that achieved following the administration of 30 mg/kg, i.p. to rats. As such, in some embodiments the effective dose (−)-E-OH-NT for the treatment of depression is the dosage required to achieve the plasma concentration achieved when (−)-E-10-OH-NT (30 mg/kg, i.p.) is administered to rats.

Dosage amounts of (−)-E-OH-NT for treating depression will typically be in the range of from about 0.0001 or 0.001 or 0.01 mg/kg/day to about 0.1 or 1.0 or 2.0 or 2.5 or 5.0 or 10.0 or 20.0 or 25.0 or 50.0 or 75.0 or 100 mg/kg/day with an expected dose of about 5 mg/day to about 500 mg/day, but may be higher or lower. It is believed that oral doses of (−)-E-10-OH-NT of between about 10 mg/day to about 20 or 25 or 30 or 35 or 40 or 45 or 50 or 60 or 70 or 80 or 90 or 95 or 100 or 150 or 200 or 250 or 300 mg/day will be effective in treating depression. Accordingly, some embodiments involve the administration of an oral dosage of (−)-E-10-OH-NT between about 10 mg/day to about 100 mg/day.

Further, based on prior reports of the pharmacokinetic properties of E-10-OH-NT and its enantiomers, it is believed depression can be treated with a plasma level of the enantiomers of E-10-OH-NT of between about 140 nM and about 220 nM. Based on the reported pharmacokinetic properties of (−)-E-10-OH-NT, it is believed that depression can be treated with a plasma level of (−)-E-10-OH-NT between about 140 nM and 220 nM and that these plasma levels can be achieved by administering between about 30 mg/day to about 35 or 40 or 45 or 50 or 55 or 60 or 65 or 70 or 75 or 80 or 85 or 90 or 95 or 100 or 150 or 200 or 250 or 300 mg/day. Accordingly, certain embodiments involve the administration of (−)-E-10-OH-NT at the daily dose required to achieve a plasma level of between about 140 nM and 220 nM. In other embodiments the dosage of (−)-E-10-OH-NT is between about 30 mg/day and about 100 mg/day.

In the context of combination therapy, the proper dosage of the combined agents will be readily ascertainable by a skilled artisan based on the above disclosed dosages for (−)-E-10-OH-NT and long established criteria for the particular indication. By way of general guidance, where a cannabinoid, opioid and/or other agent is used in combination with the E-10-OH-NT compositions described herein, typically the dosage may range from about 0.01 to about 100 mg/kg/day of the cannabinoid, opioid and/or other active compound and about 0.001 to about 100 mg/kg/day of the E-10-OH-NT composition described herein. In certain embodiments, the dosage may be about 0.1 to about 10 mg/kg/day of the cannabinoid, opioid and/or other active compound and about 0.01 to about 10 mg/kg/day of the E-10-OH-NT compositions described herein and in other embodiments, the daily dosage may be about 1.0 mg of the cannabinoid, opioid and/or other active compound and about 0.1 mg of the E-10-OH-NT compositions described herein. Alternatively, when the E-10-OH-NT compositions described herein are combined with a cannabinoid compound (e.g., Δ⁹-tetrahydrocannabinol or cannabidiol), an opioid compound (e.g., morphine) and/or an other agent and the combination is administered orally, generally the dosage may range from about 15 to about 200 mg of the cannabinoid, opioid and/or other agent, and about 0.1 to about 4 mg of the E-10-OH-NT compositions described herein.

8.7 Advantageous Properties of (−)-E-10-OH-NT

As a result of the untoward side effects discussed herein, AT and NT are no longer used as first line therapies for depression or pain. Surprisingly, the E-10-OH-NT compositions described herein have appreciably reduced untoward side effects.

For example, it has been known for several decades that (1) centrally active drugs with high affinities for H₁ histaminergic receptors can induce weight gain, (2) some antipsychotic drugs are potent H₁ histaminergic receptor antagonists, and (3) antipsychotic drugs can induce weight gain. Interestingly, other psychoactive compounds with high H₁ histaminergic receptor affinities, for example AT (Altamura et al., 1989), have been associated with significant weight gain. It has been shown that atypical anti-psychotics such olanzapine, and clozapine, as well as tricyclic and tetracyclic antidepressants such as AT and mirtazapine, respectively, which are potent H₁ histaminergic antagonists, have a high propensity to induce weight gain (Wirshing, 1999). Additionally, weight gain mediated by H₁ histaminergic antagonism is listed as a common and well-known side effect of short term and long-term treatment with tricyclic antidepressants, primarily as a result of excessive appetite (Richelson, 2001; Khawam et al., 2006; Deshmukh et al., 2003). The mechanism(s) by which H₁ histaminergic antagonism might induce weight gain are currently unknown, although prior studies have amply demonstrated that H₁ histaminergic receptor antagonism increases feeding in rodents whereas H₂ histaminergic antagonism does not (Sakata et al., 1988; Fukagawa et al., 1989). Additionally, depletion of neuronal histamine increases feeding (Menon et al., 1971; Sakai et al., 1995). Finally, H₁ knockout mice are relatively resistant to the anorectic actions of leptin, and are prone to obesity when placed on high-fat diets (Masaki et al., 2001a, Masaki et al., 2001b). Taken together, these results imply that H₁ histaminergic receptors modulate feeding behavior via a leptin-dependent mechanism. The study by Kroeze and collaborators (Kroeze et al., 2003) demonstrated that H₁ histaminergic receptor affinity is significantly correlated with short-term weight gain when a large number of typical and atypical anti-psychotic drugs are examined. These results imply that anti-psychotic drugs with relatively high H₁ histaminergic receptor affinities are likely to induce short-term weight gain.

As explained in detail in Example 7, quite unexpectedly, the affinity of (−)-E-OH-NT for the H₁ histaminergic receptor was reduced 220-fold and 40-fold when compared to the H₁ histaminergic affinity of AT and NT, respectively. Based on these in vitro results, it is expected that (−)-E-OH-NT will produce less weight gain than the AT and NT.

Based on the affinity of (−)-E-10-OH-NT for the H₁ histamine receptor, it is also expected that treatment with the E-10-OH-NT compositions described herein would produce less sedation than AT and NT. Indeed, as discussed in more detail in Example 8, using the rat rotarod assay commonly used to assess sedative effects of drugs, the magnitude of the effects of AT (30 mg/kg, i.p.) and NT (30 mg/kg, i.p.) on rotarod performance were similar, with peak deficits of 51±20% and 60±14%, at 30 minutes, respectively. However, no impairment in rotarod performance was observed with (−)-E-10-OH-NT (30 mg/kg, i.p.). These results were confirmed in Example 17. These data demonstrate that (−)-E-10-OH-NT is significantly less sedating than AT or NT.

An additional advantageous property of (−)-E-10-OH-NT is its reduced inhibition of key drug metabolizing enzymes. The metabolic conversion of drugs generally is enzymatic. The enzyme systems involved in the biotransformation of drugs are mainly localized in the liver, although every tissue examined has some metabolic activity. Following nonparenteral administration of a drug, a significant portion of the dose may be metabolically inactivated in either the liver or the intestines before it reaches the systemic circulation. This first-pass metabolism significantly limits the oral bioavailability of highly metabolized drugs. (Benet et al., 1995). The cytochrome P450 enzyme family is the major catalyst of drug biotransformation reactions. This superfamily of enzymes catalyzes a wide variety of oxidative and reductive reactions and has activity towards a chemically diverse group of substrates. Key cytochrome P 450 enzymes include CYP2D6 and CYP2C19. Alterations in the activity of these enzymes, either due to polymorphism or through inhibition, leads to undesirable clinical consequences (see Ingelman-Sundberg et al., 1999).

As discussed in detail in Example 10, both AT and NT inhibit CYP2D6 and CYP2C19, and indeed these agents are contraindicated for use with medications which are metabolized by these enzymes. Surprisingly, however, neither CYP2D6 nor CYP2C19 were inhibited by (−)-E-10-OH-NT (see, Example 10). It is therefore expected that (−)-E-10-OH-NT would engender fewer undesirable clinic consequences when compared to AT and NT, and could be used effectively in combination with drugs metabolized by these cytochrome P450 isoenzymes, and contraindicated for use with AT or NT therapy.

Another advantage to the use of (−)-E-10-OH-NT arises from its reduced affinity for muscarinic receptors. While the reduced muscarinic affinity of racemic (+)-E-10-OH-NT has been reported in the art, there are no reports of the affinity of the (−) enantiomer for the muscarinic receptors in general or for the specific muscarinic receptor subtypes. The affinity of E-10-OH-NT for muscarinic receptors in vitro was only one-eighteenth of the affinity of NT for these receptors (Nilvebrant et al. 1991; Waegner et al., 1984). In healthy individuals, NT decreased saliva flow to a significantly greater extent than either E-10-OH-NT or placebo (Nordin et al., 1987a). Additionally in an ultrarapid hydroxylator of NT treated with very high doses of NT, the plasma concentration of unconjugated 10-OH-NT was very high without any sign of anticholinergic adverse effects. These results show that 10-OH-NT metabolites have much less anticholinergic effect than AT and NT. Consistent with these results, the data explained in detail in Example 12, establish that (−)-E-10-OH-NT has a lower affinity for the four classes of muscarinic receptors, M₁, M₂, M₃, M₄, than either AT or NT.

An additional advantage of (−)-E-10-OH-NT over AT and NT is its reduced effect on GI transit (see, Example 13). Numerous signaling mechanisms, mediated by a variety of neurotransmitters within the enteric nervous system, are known to play a major role in physiological control of gastrointestinal function. The mouse gastro-intestinal transit (GIT) assay is used as preclinical marker for potential GI liability of selected compounds. As explained in detail in Exhibit 13, (−)-E-10-OH-NT showed markedly lower inhibition of GIT than AT and NT.

The use of AT and NT is associated with cardiotoxicity, including arrhythmias. The blocking of the cardiac K⁺ channel (Ikr) has been linked to drug-induced long QT syndrome (LQT), which can lead to Torsades de Pointes, a life threatening form of arrhythmia, and subsequent ventricular fibrillation (see, Pearlstein et al., 2003). The risk of QT prolongation and associated arrhythmia by tricyclic antidepressants (TCAs) corresponds at the cellular level with pharmacological inhibition of native cardiac delayed rectifier potassium (K⁺) channels and current carried by the cloned α subunit mediating the rapid delayed rectifier current—hERG. TCAs such as imipramine and AT inhibit hERG channels at clinically relevant concentrations (Witchel et al., 2002). A further advantage of (−)-E-10-OH-NT, as explained in detail in Example 11, is that it has a significantly lower activity at the hERG channel than AT and NT. Based on this result, it is expected that treatments utilizing (−)-E-10-OH-NT will yield reduced risk of QT prolongation, Torsade de Pointes and other arrhythmic-related adverse effects compared to AT and NT.

The tricyclic antidepressants also produce blood pressure effects due to their interaction with the α₁ adrenergic receptors. The blockade of α₁ adrenergic receptors, which occurs notably with doxepin, nefazodone, AT and clomipramine, is responsible for orthostatic hypotension, dizziness and reflex tachycardia (Hamon & Bourgoin, 2006). An advantage of (−)-E-10-OH-NT, as described in detail in Example 9, is its weaker affinity at the α₁ₐ and α₁ₐ adrenergic receptors compared to AT and NT. Based on these in vitro data, it is expected that (−)-E-10-OH-NT will be less likely to produce orthostatic hypotension, dizziness and reflex tachycardia.

An additional advantageous property of (−)-E-10-OH-NT arises from its lower affinity for α₂ adrenergic receptors (see, Example 9). It is well established that α₂ adrenergic receptor agonists produce analgesia (Ongioco et al., 2000; Asano et al., 2000; Hall et al., 2001). The ability of α₂ adrenergic receptor antagonists to reverse α₂ adrenoceptor-mediated antinociception is also well known (Kalso et al., 1991; Millan and Colpaert, 1991; Pertovaara et al., 1990). The α₂ adrenoceptor antagonists RX821002 ([2-(2-methoxy-1,-4-benzodioxan-2-yl)-2-imidazoline]) at doses up to 3 mg/kg when administered by itself subcutaneously did not produce significant inhibition of acetic acid-induced abdominal constriction (pain assay). Under the same experimental conditions, the α₂ adrenoceptor agonist clonidine, on the other hand, yielded a dose-dependent inhibition of abdominal constriction over the dose range 0.01 up to 1.0 mg/kg (s.c.) and at the highest dose level, it totally abolished the abdominal constriction response, thus affording 100% protection against the algogenic stimulus. Additionally, the antinociceptive effects induced by clonidine at 0.3 and 1.0 mg/kg were markedly attenuated by concurrent subcutaneous administration of RX821002 (1 mg/kg) emphasizing that the clonidine response in this test involved α₂ adrenoceptors (Gray et al., 1999).

Results on the effect of α₂ adrenergic receptor antagonists in pain response have been, however, very variable. One explanation to the variability in the α₂ adrenoceptor antagonist-induced actions in various pain tests is the fact that these drugs may mediate some of their behavioral effects via other receptor types (Dennis et al., 1980; Virtanen et al., 1989). For example, the results by Kauppila and collaborators (Kauppila et al., 1998) indicate that the effect of atipamezole, an α₂ adrenoceptor antagonist, on nocifensive behavior varies from facilitation to suppression depending on several experimental parameters. From these reports, antagonism at the α₂ adrenergic receptor does not appear to be necessary for analgesic activity.

As explained in more detail in Example 9, (−)-E-10-OH-NT exhibits significantly lower affinity than AT and NT for the adrenergic receptors α₂ₐ, α_{2b}, and α_{2c}. Based on this data, it is expected that (−)-E-10-OH-NT will be superior to AT and/or NT with respect to potential side effects: the analgesic effects of (−)-E-10-OH-NT may not be offset by an interaction with α₂ adrenergic receptors, while the analgesic effects of AT and NT may be reduced by interaction with α₂ adrenergic receptors.

8.8 Kits

The (−)-E-10-OH-NT compounds and/or pharmaceutical salts thereof described herein may be assembled in the form of kits. In some embodiments, the kit provides the compounds(s) and reagents to prepare a composition for administration. The composition may be in a dry or lyophilized from, or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch or inhalant.

The kits may include other therapeutic agents for use in conjunction with the compounds described herein. In some embodiments, the therapeutic agents may be provided in a separate form, or mixed with the compounds described herein.

Kits will include appropriate instructions for preparation and administration of the composition, side effects of the compositions, and any other relevant information. The instructions may be in any suitable format; including, but not limited to, printed matter, videotape, computer readable disk, or optical disk.

### 9. EXAMPLES

The following working examples, which are intended to be illustrative and not limiting, highlight various features and advantages of the various E-10-OH-NT compositions and methods described herein.

### Example 1

### Chiral-Specific Synthesis of (+)-E-10-OH-NT

With reference to FIG. 1A, enantiomerically pure (+)-E-10-OH-NT was synthesized as follows.

Synthesis of Compound 2. A solution of bromine (35.0 mL, 679 mmol, 1.40 eq) in carbon tetrachloride (200 mL) was added drop wise into a stirred mixture of 5-Oxo-10,11-dihydro-dibenzo[a,b]cycloheptane (compound 1; 100.0 g, 485 mmol, 1.00 eq) and carbon tetrachloride (400 mL) at room temperature. An additional 200 mL carbon tetrachloride was added to facilitate stirring, and the mixture was stirred for 90 min at room temperature.

The mixture was filtered, rinsed with carbon tetrachloride (200 mL) and dried to give 170 g of a tan solid (90% yield). This solid (170 g, 464 mmol, 1.00 eq) was combined with sodium hydroxide (55.7 g, 1.39 mol, 3.00 eq) and the mixture refluxed in methanol (2 L) for 2 hours. The hot solution was filtered and the solid dissolved in dichloromethane (400 mL) and washed with water (300 mL) and brine (200 mL). The organics were concentrated and dried to give 96.24 g of a pale orange solid. The filtrate was left to cool to room temperature overnight and more product precipitated out. The solid was filtered to give 22.2 g of light orange crystals. Combined yield: 88%. ¹H NMR (400 MHz, CDCl₃) δ 8.16 (d, 1H), 7.93 (m, 2H), 7.79 (s, 1H), 7.68-7.51 (m, 4H), 7.44 (d, 1H).

Synthesis of Compound 3. Potassium t-butoxide (62.7 g, 559 mmol, 1.40 eq) was added to a mixture of compound 2 (114 g, 400 mmol, 1.00 eq) and piperidine (79.1 mL, 800 mmol, 2.00 eq) in t-butanol (900 mL). The mixture was refluxed for 60 minutes, cooled to room temperature and concentrated in vacuo to near dryness. The crude product was dissolved in ethyl acetate (400 mL) and the resulting organic mixture was washed with water (300 mL) and brine (200 mL). The organics were concentrated and the resultant crude oil stirred in methanol (500 mL) to precipitate a yellow solid, which was filtered and dried to give 63.6 g of the desired product (55% yield). ¹H NMR (400 MHz, CDCl₃) δ 8.08 (dd, 1H), 7.87 (dd, 1H), 7.82 (dd, 1H), 7.58 (dt, 1H), 7.52-7.45 (m, 2H), 7.41 (m, 1H), 7.33 (dt, 1H), 6.38 (s, 1H), 2.89 (brs, 4H), 1.74 (m, 4H), 1.61 (brm, 2H); Mass Spectral Analysis m/z=290.1 (M+H)⁺.

Synthesis of Compound 4. A solution of cyclopropylmagnesium bromide in tetrahydrofuran (0.50 M in THF, 531 mL, 266 mmol, 1.21 eq) was added drop wise, under nitrogen, to a cooled (ice/water bath) solution of compound 3 (63.6 g, 220 mmol, 1.00 eq) dissolved in tetrahydrofuran (100 mL). The reaction mixture was stirred at room temperature for 1 hour. An additional portion of cyclopropylmagnesium bromide (0.50 M in THF, 100 mL, 50 mmol, 0.23 eq) was added and the reaction stirred for an additional hour. The reaction mixture was concentrated to near dryness, diluted with dichloromethane (600 mL) and washed with water (800 mL) and brine (300 mL). The organics were concentrated and dried to give 70.1 g of a yellow-orange sticky oil (96% yield). The crude product was used for the next step without further purification. Mass Spectral Analysis m/z=332.2 (M+H)⁺.

Synthesis of a Mixture of Compounds 5 and 6. The Z- and E-geometrical isomers of 5-[3-Bromo-propylidene]-5,11-dihydro-dibenzo[a,d]cyclohepten-10-one, compounds 5 and 6, respectively, were produced by refluxing a solution of compound 4 (70.0 g, 211 mmol, 1.00 eq) in 48% aqueous hydrobromic acid (250 mL) and acetic acid (250 mL) for 16 hours. The reaction mixture was cooled to room temperature, diluted with water (200 mL) and extracted 3 times with diethyl ether (500 mL total). The organics were combined and stirred in a large beaker, adding saturated sodium bicarbonate (300 mL) carefully until bubbling stopped. The layers were separated and the organics washed with saturated sodium bicarbonate (200 mL) and brine (150 mL) and concentrated to yield a crude semi-solid. Before the crude product was run over silica, 11 g of insoluble tan solid was filtered to give a mixture of the geometrical E- (compound 6) and Z- (compound 5) isomers (ratio of compound 6/compound 5=87/13 by ¹H NMR). The filtrate was purified by a silica gel plug using 5-10% ethyl acetate/hexanes gradient and the purified product was triturated in 10% ethyl acetate/hexanes to give 19 g of a tan solid corresponding to a mixture of geometrical isomers 5 and 6 (6/5=55/45 by ¹H NMR). This mixture of isomers was recrystallized with 1:1 benzene:hexane to give 8.6 g 76% of pure compound 6. This material was then combined with the previously isolated mixture (11 g) of geometrical isomers 5 and 6 (6/5=87/13 by ¹H NMR) and crystallized in 1:1 benzene:hexanes to give 12.7 g of a light orange solid corresponding to a mixture of geometrical isomers 5 and 6 (6/5=91/9 by ¹H NMR). ¹H NMR (400 MHz, CDCl₃) δ 8.11 (dd, 1H), 7.50 (m, 2H), 7.36 (m, 2H), 7.24 (m, 3H), 6.17 (m, 1H), 4.48 (d, 1H), 3.78 (d, 1H), 3.47 (m, 2H), 2.86-2.66 (m, 2H).

Synthesis of (+)-E-5-(γ-bromopropylidene)-10,11-dihydro-10-hydroxy-5H-dibenzo[a,d]-cycloheptene, compound (+)-7. A solution of compound 6 (2.00 g, 6.11 mmol, 1.00 eq) in tetrahydrofuran (25 mL) was added drop wise to a solution of borane-dimethyl sulfide complex (0.326 mL, 3.67 mmol, 0.60 eq) and (7αR)-3-methyl-1,1-diphenylperhydro-3-bora-2-oxapyrrolizine (1.02 g, 3.67 mmol, 0.60 eq) in tetrahydrofuran (75 mL) at −20° C. (dry ice/acetonitrile bath). The reaction was stirred at −20° C. for 90 minutes, then at room temperature for 45 minutes. Additional portions of borane-dimethyl sulfide complex (0.326 mL, 3.67 mmol, 0.60 eq) and (7αR)-3-methyl-1,1-diphenylperhydro-3-bora-2-oxapyrrolizine (1.02 g, 3.67 mmol, 0.60 eq) were added and the reaction stirred for an additional 30 minutes at room temperature. The reaction mixture was cooled to 0° C. in an ice/water bath and methanol (15 ml) added drop-wise. The mixture was stirred for 30 minutes at room temperature, cooled again to 0° C. and saturated sodium bicarbonate (20 mL) added. The mixture was stirred at room temperature for 30 minutes, concentrated and partitioned between dichloromethane (75 mL) and water (50 mL). The organics were concentrated and the crude product purified by flash silica gel column chromatography using a 10-30% ethyl acetate/hexanes gradient to give 950 mg of an off-white semi-solid in 47% yield. ¹H NMR (400 MHz, CDCl₃) δ 7.50-7.10 (m, 8H), 6.00-5.84 (m, 1H), 5.09 (brm, ⅔H), 4.85 (brm, ⅓H), 3.65-3.43 (m, 3H), 3.05 (m, 1H), 2.78-2.62 (m, 2H), 1.64 (d, 1H).

Synthesis of (+)-E-10-OH-NT, compound (+)-8. (+)-E-10-OH-NT was synthesized by heating a solution of (+)-7 (0.95 g, 2.6 mmol, 1.00 eq) and methyl amine (40% by weight in water, 7.00 mL, 81 mmol, 31 eq) in acetonitrile (10 mL) at 60° C. for 16 hours in a pressure vessel. The reaction was concentrated to near dryness and purified by flash silica gel column chromatography using a 5-9% methanol/chloroform gradient (plus 1% ammonium hydroxide) to give 650 mg of a light orange foam in 90% yield. Because there was some Z-isomer present, i.e., (+)-Z-10-OH-NT, the impure free base (650 mg, 2.33 mmol, 1.00 eq) was dissolved in acetonitrile and filtered to remove any insoluble particles. The filtrate was concentrated, dissolved in acetonitrile (15 mL) and maleic acid (324 mg, 2.79 mmol, 1.20 eq) was added. The mixture was stirred for 30 minutes and a solid precipitated out. The mixture was concentrated, dried and recrystallized from isopropanol (100 mL) to give 535 mg of a white crystalline solid, i.e., (+)-E-10-OH-NT as the maleate salt. ¹H NMR (400 MHz, DMSO-d₆) δ 8.29 (brs, 2H), 7.50-7.12 (m, 8H), 6.02 (s, 2H), 5.90 (brm, ⅓H), 5.71 (brm, ⅔H), 5.31 (brs, ⅓H), 5.08 (brs, ⅓H), 4.60 (brs, ⅓H), 3.36 (m, 1H), 3.06-2.86 (brm, 3H), 2.52 (d, 3H), 2.36 (brm, 2H). Mass Spectral Analysis m/z=280.1 (M+H)⁺. Chiral LC Analysis: 99.4% chiral purity. Column: Chromtech CHIRAL-AGP 150×4.0 mm, 5μ. Flow: 1.0 mL/min. Mobile Phase: 80% 20 mM sodium phosphate pH 6.0, 20% IPA. Detector: UV at 240 nm. Peak Retention Time: Peak 1 [(+)-E-10-OH-NT]=5.4 min. Peak 2 [(−)-E-10-OH-NT]=7.3 min. Elemental analysis: C₁₉H₂₁NO.C₄H₄O₄. Theory: % C, 69.86; % H, 6.37; % N, 3.54. Found: % C, 69.91; % H, 6.43; % N, 3.61. [α]_{D}²³³+27.79 (c. 10.1 mg/mL, MeOH). m.p.=180.5-182.0° C.

### Example 2

### Chiral-Specific Synthesis of (−)-E-10-OH-NT

With reference to FIG. 1A, enantiomerically pure (−)-E-10-OH-NT was synthesized as follows.

Synthesis of (−)-E-5-(γ-bromopropylidene)-10,11-dihydro-10-hydroxy-5H-dibenzo[a,d]-cycloheptene, compound (−)-7. A solution of compound 6 (2.50 g, 7.64 mmol, 1.00 eq) in tetrahydrofuran (25 mL) was added drop wise to a solution of borane-dimethyl sulfide complex (0.455 mL, 5.12 mmol, 0.67 eq) and (7αS)-3-methyl-1,1-diphenylperhydro-3-bora-2-oxapyrrolizine (1.42 g, 5.12 mmol, 0.67 eq) in tetrahydrofuran (75 mL) at −20° C. (dry ice/acetonitrile bath). The reaction was stirred at −20° C. for 60 minutes, then at room temperature for 2 hours. Additional portions of borane-dimethyl sulfide complex (0.455 mL, 5.12 mmol, 0.67 eq) and (7αS)-3-methyl-1,1-diphenylperhydro-3-bora-2-oxapyrrolizine (1.42 g, 5.12 mmol, 0.67 eq) were added and the reaction was stirred for an additional 30 minutes at room temperature. The reaction mixture was cooled to 0° C. in an ice/water bath and methanol (15 ml) added drop-wise. The mixture was stirred for 30 minutes at room temperature, cooled again to 0° C. and saturated sodium bicarbonate (20 mL) added. The mixture was stirred at room temperature for 30 minutes, concentrated and partitioned between dichloromethane (75 mL) and water (50 mL). The organics were concentrated and the crude product purified by flash silica gel column chromatography using a 10-30% ethyl acetate/hexanes gradient to give 2.45 g off-white sticky foam in 97% yield. ¹H NMR (400 MHz, CDCl₃) δ 7.50-7.10 (m, 8H), 6.00-5.84 (m, 1H), 5.09 (brm, ⅔H), 4.85 (brm, ⅓H), 3.65-3.43 (m, 3H), 3.05 (m, 1H), 2.78-2.62 (m, 2H), 1.64 (d, 1H).

Synthesis of (−)-E-10-OH-NT, compound (−)-8. (−)-E-10-OH-NT was synthesized by heating a solution of (−)-7 (2.45 g, 6.7 mmol, 1.00 eq) and methyl amine (40% by weight in water, 25.0 mL, 290 mmol, 43 eq) in acetonitrile (35 mL) at 60° C. for 5 hours in a pressure vessel. The reaction was concentrated and purified by a silica gel plug using a 5-9% methanol/chloroform gradient (plus 1% ammonium hydroxide). Because there was some Z isomer present, i.e., (−)-Z-10-OH-NT, the impure free base was dissolved in dichloromethane, filtered to remove any insoluble particles, concentrated and dried to give 1.9 g of a light yellow foam in quantitative yield. The free base (1.37 g, 4.9 mmol, 1.00 eq) was dissolved in acetonitrile (40 mL) and maleic acid (0.654 g, 5.64 mmol, 1.15 eq) is added. The mixture was stirred for 60 minutes and a solid precipitated out. The mixture was concentrated, dried and recrystallized from isopropanol (70 mL) to give 820 mg of pale orange needles of (−)-E-10-OH-NT as the maleate salt. ¹H NMR (400 MHz, DMSO-d₆) δ 8.29 (brs, 2H), 7.50-7.12 (m, 8H), 6.02 (s, 2H), 5.90 (brm, ⅓H), 5.71 (brs, ⅔H), 5.31 (brs, ⅓H), 5.08 (brs, ⅓H), 4.60 (brs, ⅓H), 3.36 (m, 1H), 3.06-2.86 (brm, 3H), 2.52 (d, 3H), 2.36 (brm, 2H). Mass Spectral Analysis m/z=280.1 (M+H)⁺. Chiral LC Analysis: 98.6% chiral purity. Column: Chromtech CHIRAL-AGP 150×4.0 mm, 5μ. Flow: 1.0 mL/min. Mobile Phase: 80% 20 mM sodium phosphate pH 6.0, 20% IPA. Detector: UV at 240 nm. Peak Retention Time Peak 1 [(+)-E-10-OH-NT]=6.1 min. Peak 2 [(−)-E-10-OH-NT]=8.6 min. Elemental analysis: C₁₉H₂₁NO.C₄H₄O₄. Theory: % C, 69.86; % H, 6.37; % N, 3.54. Found: % C, 69.53; % H, 6.44; % N, 3.57. [α]_{D}^{23.4}=−24.12 (c. 10.7 mg/mL, MeOH). m.p.=177.5-179.0° C.

### Example 3

### (−)-E-10-OH-NT Succinate has Improved Properties as Compared to (−)-E-10-OH-NT Maleate

Preparation of (−)-E-10-OH-NT Succinate. The succinate salt of (−)-E-10-OH-NT was prepared by dissolving 9.15 g of (−)-8 (free base, 88.6% E isomer, 11.4% Z isomer) in isopropanol (40 mL) and adding a solution of succinic acid (4.25 g, 1.10 eq) in isopropanol (90 mL). A minor amount of product crystallized out of solution after 2 days, so the mixture was concentrated to a 20 mL mixture and the resulting solid was filtered to give 11 g of succinate salt of (−)-E-10-OH-NT which was 94% E isomer and 6% Z isomer. The solid was recrystallized twice from acetonitrile (400 mL and 300 mL) to give 6.78 g light orange crystals (99.2% E isomer, 0.8% Z isomer). The resultant succinate salt is crystalline and non-hydroscopic in nature, and has a higher solubility in saline (18 mg/ml) than the maleate salt (4.4 mg/ml).

### Example 4

### (+)-E-10-OH-NT and (−)-E-10-OH-NT and are Potent Selective Inhibitors of Norepinephrine Reuptake

Protocol. The binding affinities of AT, NT, (+)-E-10-OH-NT and (−)-E-10-OH-NT for the norepinephrine (NE) transporter, the serotonin (5HT) transporter and dopamine (DA) transporter were determined in competitive binding assays with radiolabeled ligands. For the NE transporter binding assay, [³H]nisoxetine (1.0 nM) was incubated with various concentrations of test compounds for 2 hours at 4° C. with membranes prepared from Chinese hamster ovary cells (CHO) cells heterologously expressing the cloned human NE transporter (hNET). Bound radioactivity was determined by scintillation spectroscopy. Non-specific binding was defined as the amount of binding that occurred in the presence of 1.0 μM desipramine. The Kᵢ values of the various test compounds were determined using standard methods.

For the 5HT transporter binding assay, [³H]imipramine (2.0 nM) was incubated in the presence of various concentrations of test compounds for 1 hour at 22° C. with membranes prepared from CHO cells heterologously expressing the human serotonin transporter (hSERT). Bound radioactivity was determined by scintillation spectroscopy. Non-specific binding was defined as the amount of binding that occurred in the pressure at 10 μM imipramine. The Kᵢ values of the various test compounds were determined using standard methods.

For the DA transporter binding assay, [³H]N-[1-(2-benzo[b]thiophenyl)cyclohexyl]-piperidine ([³H]BTCP) (4.0 nM) was incubated for 2 hr at 4° C. with membranes prepared from Chinese hamster ovary (CHO) cells heterologously expressing the cloned human dopamine transporter (hDAT). Various concentrations of test compound were added and bound radioactivity was determined by scintillation spectroscopy. Non-specific binding was defined as binding that occurred in the presence of 10 μM BTCP. The Kᵢ values for the various test compounds were determined using standard methods.

The ability of AT, NT (+)-E-10-OH-NT, and (−)-E-10-OH-NT to inhibit uptake of NE, 5HT and DA was also assessed. The IC₅₀ values in NE uptake was determined by measuring the inhibition of the incorporation of [³H]-norepinephrine into rat hypothalamus synaptosomes upon incubation for 20 minutes at 37° C. The IC₅₀ values in 5HT uptake was determined by measuring the inhibition of the incorporation of [³H]-5HT into rat brain synaptosomes upon incubation for 15 min at 37° C. The IC₅₀ values in DA uptake was determined by measuring the inhibition of the incorporation of [³H]-DA into rat striatum synaptosomes upon incubation for 15 min at 37° C.

Results. The binding affinities for the various transporters are provided in Table 1, below. In Table 1, Kᵢ values are in nanomolar. Percentages are the percent inhibition of binding observed with 10 μM test compound.

The uptake inhibition data are reported in Table 2, below. In Table, 2, IC₅₀ values are in nanomolar. Percentages are the percent inhibition of uptake observed with 10 μM test compound.

TABLE 1
Monoamine Transporter Affinity Kᵢ (nM) or % inhib at 10 μM

| | DA | 5HT | NE | 5HT:NE |
|---|---|---|---|---|
| AT | 2100 | 3.8 | 40 | 0.095 |
| NT | 1000 | 37 | 7.4 | 5.0 |
| (+)-E-10-OH-NT | 25% (>1000) | 400 | 52 | 7.7 |
| (−)-E-10-OH-NT | 3% (>1000) | 660 | 44 | 15.0 |

TABLE 2
Monoamine Uptake Inhibition IC₅₀ (nM) or % inhib. at 10 μM

| | DA | 5HT | NE | 5HT:NE |
|---|---|---|---|---|
| AT | 2400 | 40 | 20 | 2.0 |
| NT | 1700 | 99 | 3.4 | 29 |
| (+)-E-10-OH-NT | 19% (>1000) | 860 | 14 | 61 |
| (−)-E-10-OH-NT | 16% (>1000) | 4400 | 31 | 140 |

The affinities of (+)-E-10-OH-NT and (−)-E-10-OH-NT for the NE transporter were approximately equal to that of AT, and within about 10-fold that of NT. Similar results were observed for the IC₅₀ values for the inhibition of NE uptake. The IC₅₀ values for the inhibition of NE uptake are lower than those previously reported for racemic E-10-OH-NT. (See Hyttel, 1980 which reported an IC₅₀ of 130 nM for NE uptake assay in mouse atria.)

While the affinities of (+)-E-10-OH-NT and (−)-E-10-OH-NT for the NE transporter were comparable to that of AT, the affinities of (+)-E-10-OH-NT and (−)-E-10-OH-NT for the 5HT transporter like the affinity of NT are significantly greater than the affinity of AT. Similar results were observed for the IC₅₀ values for the inhibition of 5HT uptake. Based on these results, (+)-E-10-OH-NT and (−)-E-10-OH-NT like NT exhibit selectively for the NE transporter versus the 5HT transporter.

As will be demonstrated in Example 14, racemic (+)-E-10-OH-NT also exhibits selective affinities for the NE and 5HT transporters that are approximately equipotent to those of the (+) and (−) enantiomers.

### Example 5

### (−)-E-10-OH-NT is an Effective Antihyperalgesic Agent

Protocol. The antihyperalgesic effectiveness of (−)-E-10-OH-NT was demonstrated in the Freund's Complete Adjuvant-induced rodent model of nociceptive inflammatory pain. For comparison, AT was tested as a positive control. Drugs were administered at 30 mg/kg, i.p. Sterile water vehicle was tested as a negative control. (−)-E-10-OH-NT was administered as either a maleate or succinate salt. AT was administered as a hydrochloride salt. Dosage amounts are based on the amount of free base. For the assay, the methods of DeHaven-Hudkins et al., 1999 were used to determine mechanical hyperalgesia in rats 24 hours after intraplantar administration of 150 μL Freund's Complete Adjuvant (FCA). To determine paw pressure thresholds, the rats were lightly restrained in a gauze wrap and pressure was applied to the dorsal surface of the inflamed and uninflamed paw with a conical piston using a pressure analgesia apparatus (Stoelting Instruments, Wood Dale, Ill.). The paw pressure threshold was defined as the amount of force (in grams) required to elicit an escape response using a cutoff value of 250 grams. Paw pressure thresholds were determined before and at specified times after drug treatment.

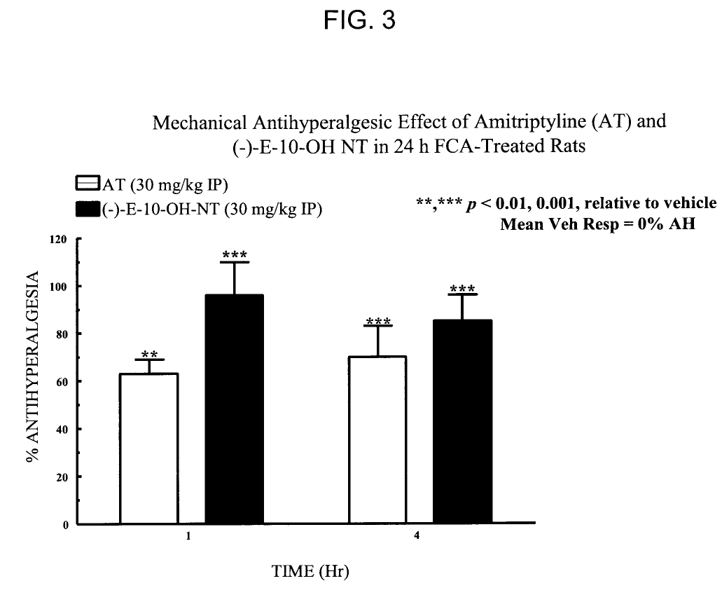

Results. The results are illustrated in FIG. 3. (−)-E-10-OH-NT (30 mg/kg, i.p.) showed robust antihyperalgesic activity comparable to that of AT (30 mg/kg, i.p.).

### Example 6

### (−)-E-10-OH-NT is Effective in Treating Non-Nociceptive Neuropathic Pain

Protocol. The antiallodynic activity of (−)-E-10-OH-NT compared to that of amitriptyline was also tested in vivo using the L5-Single Nerve Ligation model of non-nociceptive neuropathic pain as described in LaBuda and Little, 2005. The test animals were placed in a Plexiglas chamber (10 cm×20 cm×25 cm) and habituated for 15 minutes. The chamber was positioned on top of a mesh screen so that von Frey monofilaments could be presented to the plantar surface of both hindpaws. Measurement of tactile sensitivity for each hindpaw were obtained using the up/down method (Dixon, 1980) with seven Frey monofilaments (0.4, 1, 2, 4, 6, 8 and 15 grams). Each trial started with a von Frey force of 2 grams delivered to the right hindpaw for approximately 1-2 seconds and then the left hindpaw. If there was no withdrawal response, the next higher force was delivered. If there was a response, the next lower force was delivered. This procedure was performed until no response was made at the highest force (15 grams) or until four stimuli were administered following the initial response. Each test group contained 8 animals. The sham-operated control group, which were operated on but not subject to nerve ligation, contained 4 animals. All animals were tested at 60 minutes, and 240 minutes, post-administration of test compounds.

The 50% paw withdrawal threshold for each paw was calculated using the following formula: [Xth] log=[vFr] log+ky where [vFr] is the force of the last von Frey used, k=0.2249 which is the average interval (in log units) between the von Frey monofilaments, and y is a value that depends upon the pattern of withdrawal responses (Dixon, 1980). If an animal did not respond to the highest von Frey monofilament (15 grams), then the paw was assigned a value of 18.23 grams. Testing for tactile sensitivity was performed twice and the mean 50% withdrawal value assigned as the tactile sensitivity for the right and left paws for each animal.

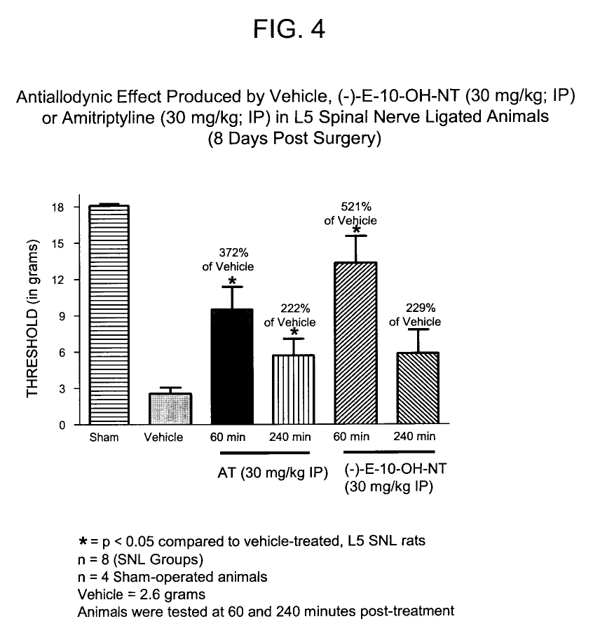

Results. The results are illustrated in FIG. 4. (−)-E-10-OH-NT was administered as either the maleate or succinate salt. AT was administered as the hydrochloride salt. Dosage amounts are based on the amount of free base. The antiallodynic activity of (−)-E-10-OH-NT (30 mg/kg, i.p.) was statistically significant over that observed for the vehicle-treated control group (vehicle-treated, L5 SNL rats), and comparable to that of AT (30 mg/kg, i.p.).

### Example 7

### (+)-E-10-OH-NT and (−)-E-10-OH-NT Bind to the H₁ Histaminergic Receptor with Weak Affinity

Protocol. The affinities of AT, NT, (+)-E-10-OH-NT and (−)-E-10-OH-NT for the H₁ histaminergic receptor were assessed in a competitive binding assay. For the assay, [³H]pyrilamine (3 nM) was incubated with various concentrations of test compound for 1 hour at 22° C. with membranes prepared from human embryonic kidney (HEK-293) cells heterologously expressing the cloned human H₁ histaminergic receptor. Bound radioactivity was determined by scintillation spectroscopy. Non-specific binding was defined as the amount of binding that occurred in the presence of 1.0 μM unlabeled pyrilamine.

Results. The binding studies indicate that the H₁ histaminergic receptor affinities of (+)-E-10-OH-NT and (+)-E-10-OH-NT were unexpectedly reduced 220-fold and 40-fold compared to the H₁ histaminergic receptor affinity of AT and NT respectively. Based on these results, it is expected that (+)-E-10-OH-NT and (−)-E-10-OH-NT would be less sedating and would produce less weight gain than AT or NT.

### Example 8

### (−)-E-10-OH-NT is Significantly Less Sedating than AT and NT In Vivo

Protocol. The rat rotarod assay is commonly used to assess sedation associated with chemical drugs. Time course experiments were performed with AT (30 mg/kg, i.p.) NT (30 mg/kg, i.p.) and (−)-E-10-OH-NT (30 mg/kg, i.p.). The rotarod was set in motion at a constant speed and the rats were placed onto the individual rotating drums of the apparatus. Once the rats were in position, the timers were set to zero and the rotarod switched to accelerating mode. The rotarod accelerated from 4 to 40 rpm over a five-minute interval. The timers automatically turned off when the rats fell from the rotating drum, recording a latency to fall in seconds. The rats had 3 training sessions, separated by at least 15 minutes, prior to drug administration. The performance score was recorded in seconds for each interval. The baseline rotarod performance score was the latency to fall off the rotarod on the third training session. Baselines must have been greater than or equal to 60 seconds duration for the rat to be included in the experiment. Animals were tested at 30, 60 and 120 minutes following administration of test compound.

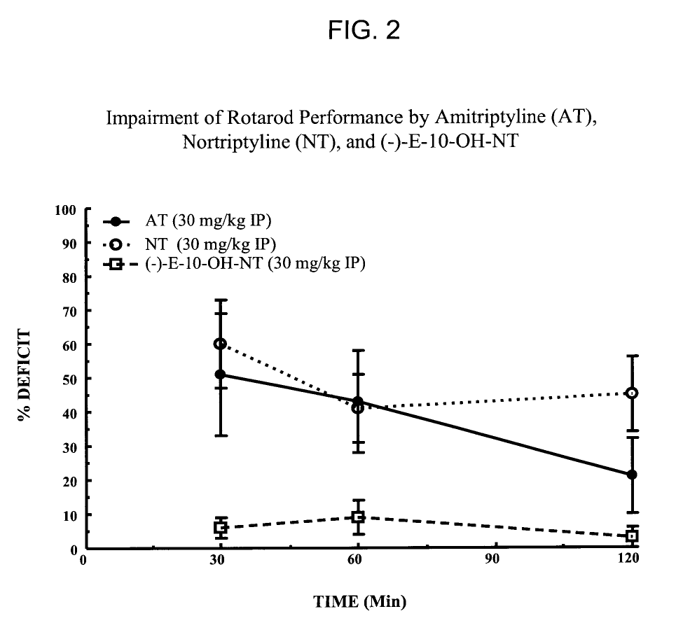

Results. The results are shown in FIG. 2. (−)-E-10-OH-NT was administered as either maleate or succinate salt. NT and AT were administered as hydrochloride salts. The dosage amounts are based on the amount of free base. Peak deficits in rotarod performance were observed at 30 minutes in rats treated with AT or NT. The magnitude of the effects of AT and NT on rotarod performance were similar, with peak deficits of 51±20% and a 60±14%, respectively. In stark contrast, no impairment of performance was observed in rats treated with (−)-E-10-OH-NT (30 mg/kg, i.p.).

### Example 9

### (+)-E-10-OH-NT and (−)-E-10-OH-NT have Low Affinity for Alpha Adrenergic Receptors

The affinities of AT, NT, (+)-E-10-OH-NT and (−)-E-OH-NT for the α₁ₐ, α_{2b}, α₂ₐ, α_{2b}, and α_{2c} adrenergic receptors was tested. Affinity for the α₁ₐ adrenergic receptor was tested using membranes prepared from rat salivary gland. [³H]Prazocine (0.06 nM) was incubated in the presence of various concentrations of test compounds with the membranes for 1 hour at 22° C. Nonspecific binding was determined in the presence of 10 μM phentolamine. Bound radioactivity was determined by scintillation spectroscopy.

Affinity for the α_{1b} adrenergic receptor was tested using membranes prepared from Chinese hamster ovary (CHO) cells heterologously expressing the cloned human receptor that were incubated with [³H]prazocine (0.15 nM) in the presence of various concentrations of test compounds for 1 hour at 22° C. Nonspecific binding was determined in the presence of 10 μM phentolamine. Bound radioactivity was determined by scintillation spectroscopy.

Affinity for the α₂ₐ adrenergic receptor was tested using CHO cells heterologously expressing the cloned human receptor that were incubated with [³H]RX821002 (1.0 nM) in the presence of various concentrations of test compounds for 1 hour at 22° C. Nonspecific binding was determined in the presence of 100 μM (−)-epinephrine. Bound radioactivity was determined by scintillation spectroscopy.

Affinity for the α_{2b} adrenergic receptor was tested using CHO cells heterologously expressing the cloned human receptor that were incubated with [³H]RX821002 (2.5 nM) in the presence of various concentrations of test compounds for 1 hour at 22° C. Nonspecific binding was determined in the presence of 100 μM (−)-epinephrine. Bound radioactivity was determined by scintillation spectroscopy.

Affinity for the α_{2c} adrenergic receptor was tested using CHO cells heterologously expressing the cloned human receptor with [³H]RX821002 (2.0 nM) in the presence of various concentrations of test compounds for 1 hour at 22° C. Nonspecific binding was determined in the presence of 100 μM (−)-epinephrine. Bound radioactivity was determined by scintillation spectroscopy.

Results. The results are tabulated in Table 3, below. Table 3, Kᵢ values are reported in nanomolar. Percentages represent the percentage of inhibition of binding observed with 10 μM test compound.

TABLE 3
Affinities at Adrenergic Receptors (Kᵢ in nM; % inhib. at 10 μM)

| | α₁ₐ | α_{1b} | α₂ₐ | α_{2b} | α_{2c} |
|---|---|---|---|---|---|
| AT | 21 | 5.9 | 570 | 8.2 | 13 |
| NT | 60 | 21 | 810 | 39 | 56 |
| (+)-E-10-OH-NT | 700 | 690 | 15% | 4200 | 35% |
| (−)-E-10-OH-NT | 3000 | 2600 | 23% | 4300 | 30% |

(+)-E-10-OH-NT and (−)-E-10-OH-NT exhibit significantly lower affinity than AT and NT for all adrenergic receptors listed, particularly at the α₂ receptors. Accordingly, it is expected that the analgesic effects of (+)-E-10-OH-NT and (−)-E-10-OH-NT may not be offset by their interaction with the α₂ adrenergic receptors while the analgesic responses of AT and NT may be offset by their interaction with the α₂ adrenergic receptors. Furthermore, the difference in affinity between (−)-E-10-OH-NT and (+)-E-10-OH-NT at the α₁ₐ and α_{1b} receptors suggests that the profile of (−)-E-10-OH-NT is superior than the (+) enantiomer.

### Example 10

### (+)-E-10-OH-NT and (−)-E-10-OH-NT do not Inhibit Cytochrome P450 Function

Protocol. The inhibitory activities of AT and NT, (+)-E-10-OH-NT and (−)-E-10-OH-NT on cytochrome P450 function were tested using the methods of Chauret et al., 2001, using 7-methoxy-4-(aminomethyl)-coumarin (MAMC) (Venhorst et al., 2000) as substrate. The source of the enzyme was microsomes containing human recombinant CYP2D6 obtained from BD Bioscience. Conversion of MAMC to 7-hydroxy-4-(aminomethyl)coumarin was measured using a PerkinElmer Fusion with a 390 nm excitation filter and a 460 nm emission filter.

CYP2C19 activity was measured using dibezylflourescein (DBF) as substrate. The source of enzyme was microsomes containing human recombinant CYP2C19 obtained from BD Biosciences (San Jose, Calif.). Conversion of DBF to fluorescein was measured using a PerkinElmer Fusion with a 485 nm excitation filter and a 535 nm emission filter.

Results. The results are tabulated in Table 4, below. In Table 4, IC₅₀ values are in nanomolar and the 95% confidence limits are shown in parentheses. Percentages reflect the percent inhibition observed with 10 μM test compound. Values reported are the average of six replicate experiments.

TABLE 4
Inhibition of CYPD26 and CYP2C19 Isozymes
(IC₅₀ in nM; % inhib. at 10 μM)

| | CYP2D6 | CYPD2C19 |
|---|---|---|
| AT | 820 (550-1200) | 3500 (2800-4400) |
| NT | 700 (560-860) | 1500 (1200-2000) |
| (+)-E-10-OH-NT | 40% ± 4.7% | 20% ± 5.8% |
| (−)-E-10-OH-NT | 37% ± 8.0% | 24% ± 6.8% |

(+)-E-10-OH-NT and (−)-E-10-OH-NT surprisingly demonstrated a significant and unexpected decrease in the inhibitory activity at the polymorphic cytochrome P450 isoenzymes CYP2D6 and CYP2C19 as compared to both AT and NT. This reduction in inhibitory activity for the (+) and (−) enantiomers of E-10-OH-NT is expected to lead to a reduction in undesirable clinical consequences when compared to AT and NT and allow for use of (+)- and (−)-E-10-OH-NT in patients taking medications contraindicated for use with AT and NT.

### Example 11

### (+)-E-10-OH-NT and (−)-E-10-OH-NT Inhibit the hERG Channel to a Lesser Extent than AT and NT

Protocol. The ability of AT, NT, (+)-E-10-OH-NT and (−)-E-10-OH-NT to block cardiac delayed rectifier potassium channels was tested with the closed α subunit indicating the rapid delayed rectifier current (hERG).

Results. The percent inhibition of hERG channels achieved with 10 μM test compound is indicated below:
AT: 68.6%
NT: 55.1%
(+)-E-10-OH-NT: 8.8%
(−)-E-10-OH-NT: 6.0%

Significantly lower activity is observed with (+)-E-10-OH-NT and (−)-E-10-OH-NT than AT and NT. Based on these results, it is expected that treatments utilizing (+)-E-10-OH-NT and (−)-E-10-OH-NT could yield a reduced risk of QT prolongation and other arrhythmic-related adverse effects as compared to treatments with AT and NT.

### Example 12

### (+)-E-10-OH-NT and (−)-E-10-OH-NT have Lower Affinities for Muscarinic Receptors than AT and NT

Protocol. The affinities of AT, NT (+)-E-10-OH-NT and (−)-E-10-OH-NT for muscarinic receptors M₁, M₂, M₃, M₄ and M₅ were determined with membranes prepared from CHO cells heterologously expressing the cloned human muscarinic receptors M₁, M₂, M₃, M₄ and M₅. For the assays, membranes and radiolabelled ligand were incubated with various concentrations of test compounds for 1 hour at 22° C. Bound radioactivity was determined by scintillation spectroscopy. Non-specific binding was defined as the amount of bound radioligand in the presence of 1.0 μM atropine. Radioligand for the M₁ receptor was [³H]pirenzepine (2 nM). Radioligand for the M₂ receptor was [³H]AF-DX384 (2 nM). Radioligand for the M₃, M₄, and M₅ receptors was [³H]4-DAMP.

Results. The affinity constants (in nanomolar) of the various different test compounds for the different receptors are tabulated in Table 5, below:

TABLE 5
Affinity for Muscarinic Receptors (Kᵢ in nM)

| | M₁ | M₂ | M₃ | M₄ | M₅ |
|---|---|---|---|---|---|
| AT | 5.6 | 66 | 8.9 | 3.6 | 3.5 |
| NT | 27 | 220 | 35 | 21 | 15 |
| (+)-E-10-OH-NT | 710 | 4200 | 820 | 390 | 280 |
| (−)-E-10-OH-NT | 410 | 2600 | 390 | 170 | 150 |

In all cases, (+)-E-10-OH-NT and (−)-E-10-OH-NT exhibited approximately equal affinities, and significantly lower affinities than both AT and NT.

### Example 13

### (−)-E-10-OH-NT Exhibits Less Inhibition of Gastrointestinal Transit than AT and NT

Protocol. The inhibitory effect of AT, NT and (−)-E-10-OH-NT were assessed in a rodent model of gastrointestinal transit. For the assay, male Swiss-Webster mice (20-25 g) were fasted overnight and treated with test compound or sterile water vehicle control 1, 2 or 4 hours before per oral administration of a charcoal meal consisting of charcoal:flour:water (1:2:8, w:w:w). Gastrointestinal transit was measured 25 minutes after the charcoal meal. (−)-E-10-OH-NT was administered as either the maleate or succinate salt; AT and NT were administered as hydrochloride salts; the dosage amounts are based on the amount of free base.

Gastrointestinal transit (GIT) was determined by removing the entire length of the small intestine and measuring how far the leading edge of the charcoal meal traveled in the small intestine. The percent GIT (% GIT) was determined by the following formula:

%GIT=[distance to charcoal leading edge (cm)/length of small intestine (cm)×100]

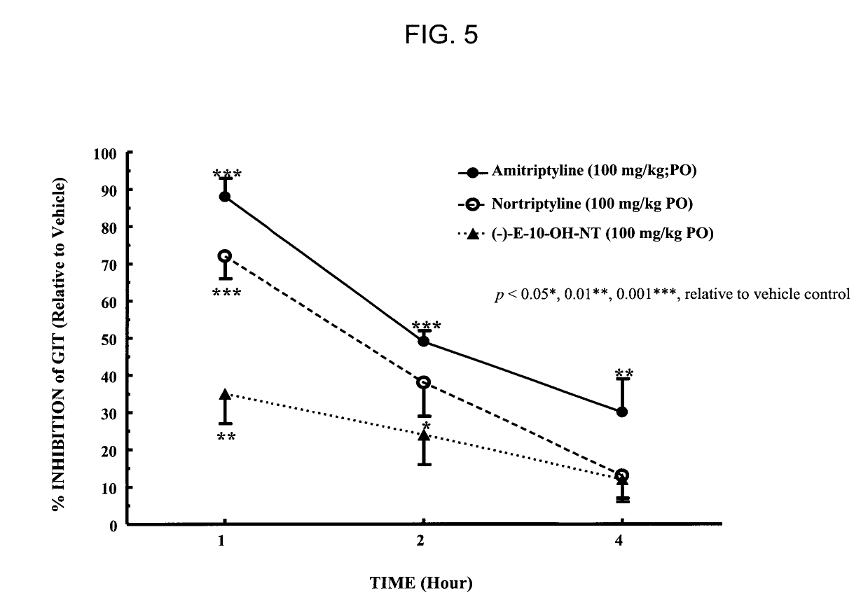

Results. The % GIT observed with the test compounds (relative to sterile water vehicle) are illustrated in FIG. 5. (−)-E-10-OH-NT showed markedly lower inhibition of transit as compared to both AT and NT.

### Example 14

### In Vitro Binding Affinities of Racemic (+)-E-10-OH-NT, (+)-E-10-OH-NT and (−)-E-10-OH-NT for Monoamine Transporters are Comparable to One Another

Protocol. The binding affinities of racemic E-10-OH-NT (“(±)-E-10-OH-NT”), (+)-E-10-OH-NT and (−)-E-10-OH-NT for the norepinephrine (NE) and serotonin (SERT) transporters were determined in competitive binding assays with radiolabeled ligands. For the NE transporter binding assay, [³H]nisoxetine (1.0 nM) was incubated with various concentrations of test compounds for 2 hours at 4° C. with membranes prepared from Chinese hamster ovary cells (CHO) cells heterologously expressing the cloned human NE transporter (hNET). Bound radioactivity was determined by scintillation spectroscopy. Non-specific binding was defined as the amount of binding that occurred in the presence of 1.0 μM desipramine. The Kᵢ values of the various test compounds were determined using standard methods.

For the 5HT transporter (SERT) binding assay, [³H]imipramine (2.0 nM) was incubated in the presence of various concentrations of test compounds for 1 hour at 22° C. with membranes prepared from CHO cells heterologously expressing the human serotonin transporter (hSERT). Bound radioactivity was determined by scintillation spectroscopy. Non-specific binding was defined as the amount of binding that occurred in the pressure at 10 μM imipramine. The Kᵢ values of the various test compounds were determined using standard methods.

Results. The binding affinities for the various transporters are provided in Table 6, below.

TABLE 6
Monoamine Transporter Affinity

| | NET Kᵢ | SERT Kᵢ |
|---|---|---|
| (±)-E-10-OH-NT | 51 nM | 420 nM |
| (+)-E-10-OH-NT | 95 nM | 361 nM |
| (−)-E-10-OH-NT | 81 nM | 617 nM |

The binding affinity of racemic (+)-E-10-OH-NT for the norepinephrine transporter was comparable to that observed for the purified enantiomers, (+)-E-10-OH-NT and (−)-E-10-OH-NT, which were approximately equal to one another. Similarly, the binding affinity of racemic (+)-E-10-OH-NT for the serotonin transporter was also comparable to that observed for both of the purified (+)-E-10-OH-NT and the (−)-E-10-OH-NT.

### Example 15

### (−)-E-10-OH-NT is More Effective in Treating Non-Nociceptive Neuropathic Pain than Both (+)-E-10-OH-NT and (+)-E-10-OH-NT

Protocol: The antiallodynic activity of (−)-E-10-OH-NT compared to that of amitriptyline, (±)-E-10-OH-NT, and (+)-E-10-OH-NT was tested in vivo using the L5-Single Nerve Ligation model of non-nociceptive neuropathic pain as described in LaBuda & Little, 2005. The E-10-OH-NT compounds were administered as either a maleate or succinate salt. AT was administered as a hydrochloride salt. Dosage amounts are based on the amount of free base. The test animals were placed in a Plexiglas chamber (10 cm×20 cm×25 cm) and habituated for 15 minutes. The chamber was positioned on top of a mesh screen so that von Frey monofilaments could be presented to the plantar surface of both hindpaws. Measurement of tactile sensitivity for each hindpaw were obtained using the up/down method (Dixon, 1980) with seven Frey monofilaments (0.4, 1, 2, 4, 6, 8 and 15 grams). Each trial started with a von Frey force of 2 grams delivered to the right hindpaw for approximately 1-2 seconds and then the left hindpaw. If there was no withdrawal response, the next higher force was delivered. If there was a response, the next lower force was delivered. This procedure was performed until no response was made at the highest force (15 grams) or until four stimuli were administered following the initial response. Each test group contained 8 animals. The sham-operated control group, which were operated on but not subject to nerve ligation, contained 4 animals. All animals were tested at 60 minutes post-administration of test compounds.

The 50% paw withdrawal threshold for each paw was calculated using the following formula: [Xth] log=[vFr] log+ky where [vFr] is the force of the last von Frey used, k=0.2249 which is the average interval (in log units) between the von Frey monofilaments, and y is a value that depends upon the pattern of withdrawal responses (Dixon, 1980). If an animal did not respond to the highest von Frey monofilament (15 grams), then the paw was assigned a value of 18.23 grams. Testing for tactile sensitivity was performed twice and the mean 50% withdrawal value assigned as the tactile sensitivity for the right and left paws for each animal.

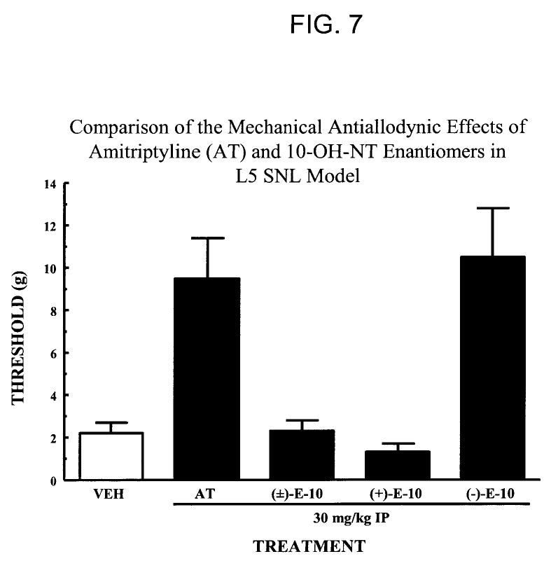

Results. The results are illustrated in FIG. 7. The antiallodynic activity of (−)-E-10-OH-NT (30 mg/kg, i.p.) was greater than that observed for the vehicle-treated control group (vehicle-treated, L5 SNL rats) (“VEH”) and comparable to that of AT (30 mg/kg, i.p.). The antiallodynic activity of (−)-E-10-OH-NT (30 mg/kg, i.p.) was also greater than that observed for both (I)-E-10-OH-NT (30 mg/kg, ip) and (−)-E-10-OH-NT (30 mg/kg, ip). The activities of the racemic mixture and the (+) enantiomer were both comparable to that of the vehicle-only control (“VEH”).

### Example 16

### (−)-E-10-OH-NT is as Effective in Treating Non-Nociceptive Neuropathic Pain as Amitriptyline Upon Oral Administration in the L5 SNL Rat Model

Protocol: The antiallodynic activity of orally administered (−)-E-10-OH-NT (maleate or succinate salt) was compared to that of amitriptyline (hydrochloride salt) in vivo using the L5-Single Nerve Ligation model of non-nociceptive neuropathic pain according to the methods described above in Example 15.

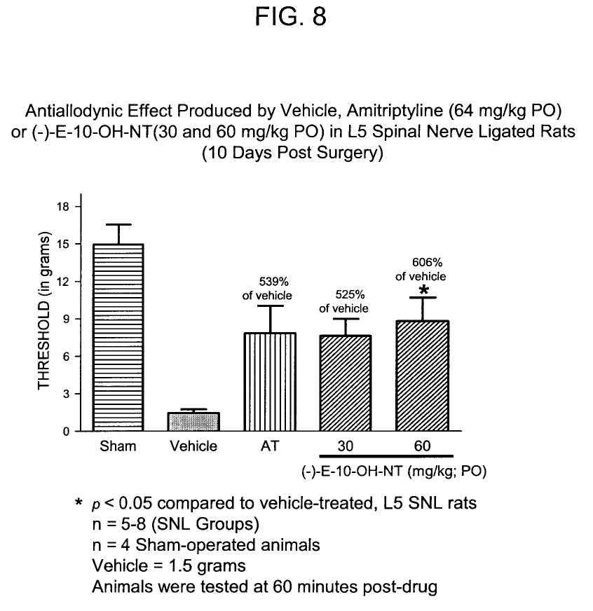

Results. The results are illustrated in FIG. 8. The antiallodynic activity of (−)-E-10-OH-NT (30 mg/kg, p.o. and 60 mg/kg p.o.) was greater than that observed for the vehicle-treated control and comparable to that of AT (30 mg/kg, i.p.). Dosage amounts are based on the amount of free base administered.

### Example 17

### FCA-Induced Hyperalgesia and Rotarod Dose-Response Curves for (−)-E-10-OH-NT and AT

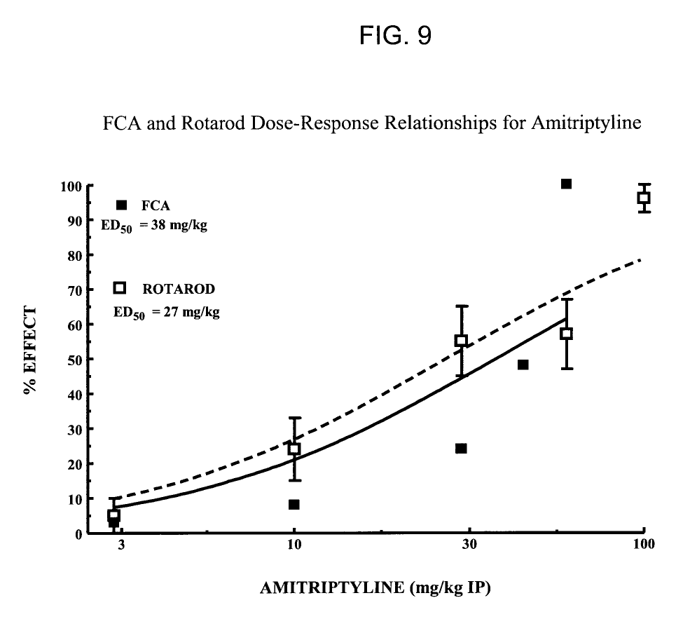

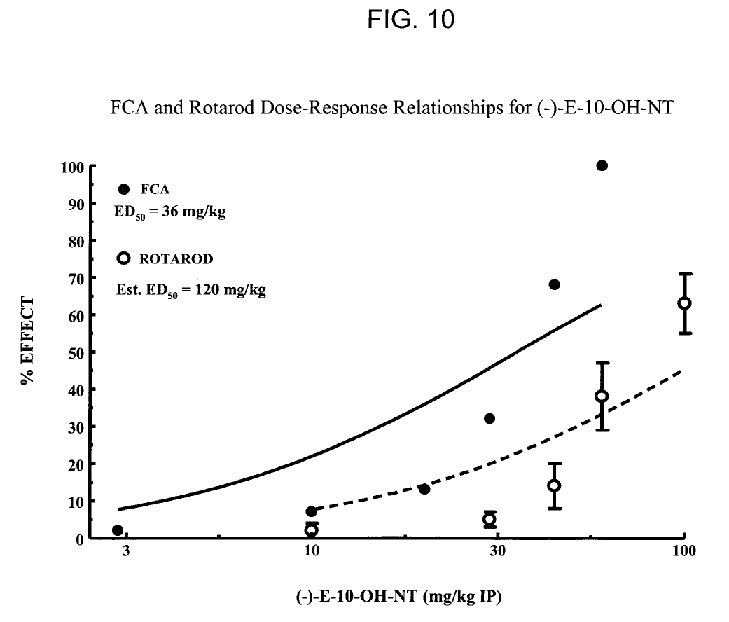

Dose-response experiments were performed with amitriptyline (hydrochloride salt) and (−)-E-10-OH-NT (maleate or succinate salt) in the rat FCA-induced hyperalgesia (Randall Selitto) assay and in the rat rotarod assay to determine a therapeutic ratio between antihyperalgesic efficacy and sedation following IP administration. Rats were treated with sterile water vehicle, AT, or (−)-E-10-OH-NT at doses up to 60 mg/kg IP (dosages are based on the amount of free base administered). Paw pressure thresholds were measured 1 h posttreatment. AT and (−)-E-10-OH-NT displayed similar potencies and efficacies for the reversal of mechanical hyperalgesia in 24 h FCA-treated rats. At 60 mg/kg IP, a high level of antihyperalgesic activity (% AH) was observed with AT and (−)-E-10-OH NT, with % AH values of 259±39 and 270±53 in rats treated with AT and (−)-E-10-OH NT, respectively (FIG. 9, FIG. 10). To estimate an ED50 value for AT and (−)-E-10-OH NT reversal of mechanical hyperalgesia, the response obtained in rats treated with 60 mg/kg IP was used as the maximal response (100%) and the responses at lower doses were calculated as a percent of the maximum effect. The ED50 values for AT and (−)-E-10-OH NT were 38 and 36 mg/kg IP, respectively (FIG. 9, FIG. 10).

Impairment of rotarod performance in rats was used to assess the degree of sedation produced by (−)-E-10-OH-NT and AT. AT dose dependently decreased rotarod performance, with an ED₅₀ value of 27 mg/kg IP and a maximal impairment of 96% observed at a dose of 100 mg/kg IP (FIG. 9). In contrast, 30 mg/kg IP (−)-E-10-OH-NT only produced a 5% impairment of rotarod performance. The estimated ED₅₀ value for (−)-E-10-OH-NT impairment of rotarod performance was 120 mg/kg IP (FIG. 10).

These data, taken together, demonstrate that (−)-E-10-OH-NT has potential to show less off-target pharmacological side effects than AT.

### Example 18

### Antihyperalgesic Activity of (−)-E-10-OH NT in the Rat FCA-Induced Hyperalgesia Assay

Protocol. The antihyperalgesic effectiveness of orally-administered (−)-E-10-OH-NT (maleate or succinate salt) was demonstrated in the Freund's Complete Adjuvant-induced rodent model of nociceptive inflammatory pain (dosages are based on the amount of free base administered). For comparison, sterile water vehicle was tested as a negative control. The methods of DeHaven-Hudkins et al., 1999 were used to determine mechanical hyperalgesia in rats 24 hours after intraplantar administration of 150 μL Freund's Complete Adjuvant (FCA). To determine paw pressure thresholds, the rats were lightly restrained in a gauze wrap and pressure was applied to the dorsal surface of the inflamed and uninflamed paw with a conical piston using a pressure analgesia apparatus (Stoelting Instruments, Wood Dale, Ill.). The paw pressure threshold was defined as the amount of force (in grams) required to elicit an escape response using a cutoff value of 250 grams. Paw pressure thresholds were determined before and at specified times after drug treatment.

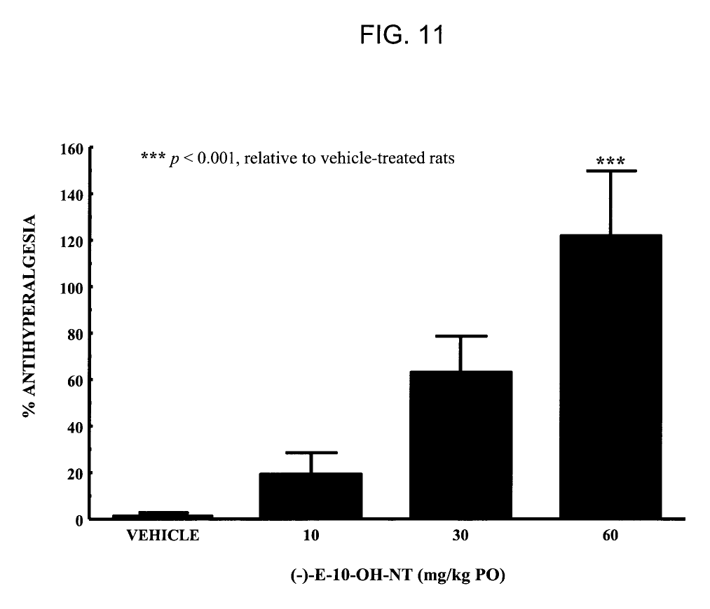

Results. The results are illustrated in FIG. 11 and demonstrate that orally-administered (−)-E-10-OH-NT provides significant activity in the rat FCA-induced hyperalgesia assay.

### Example 19

### Comparison of the Antihyperalgesic Activity of (−)-E-10-OH-NT and (+)-E-10-OH-NT in the Rat FCA-Induced Hyperalgesia Assay

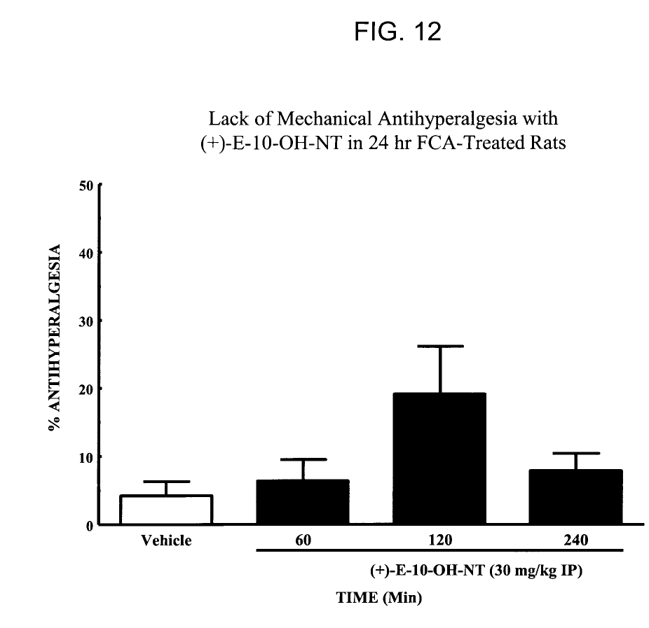

Experiments were performed to determine whether (+)-E-10-OH NT had similar antihyperalgesic potency and efficacy as (−)-E-10-OH NT in 24 h FCA-treated rats. A time course experiment was performed with 30 mg/kg IP of (+)-E-10-OH NT. In this experiment, 24 hours after FCA treatment, rats were administered either vehicle or 30 mg/kg IP of (+)-E-10-OH-NT (administered the maleate or succinate salt; the dosage amount is based on the amount of free base administered). Paw pressure thresholds were determined 1, 2, or 4 hours after administration using the methods disclosed in Examples 5 and 18. Vehicle-treated rats were tested at 1 hour post-treatment. (+)-E-10-OH NT was not antihyperalgesic at any time point tested (FIG. 12).

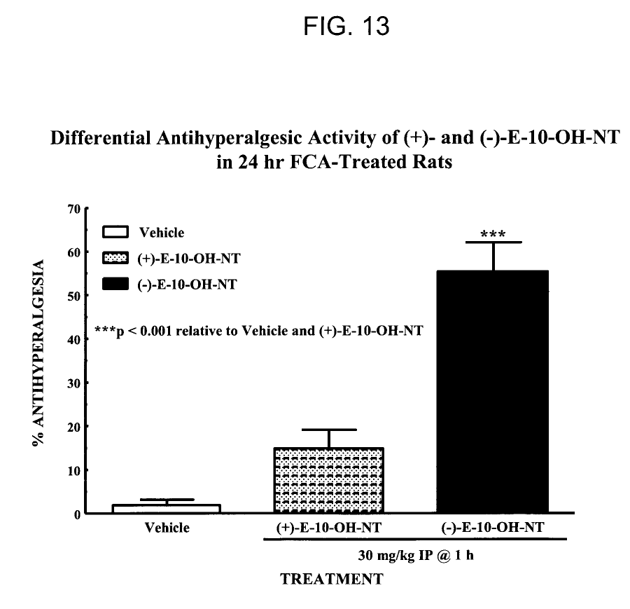

To verify these results, a 24 hour FCA-induced hyperalgesia experiment was performed in which rats were treated with vehicle or 30 mg/kg IP of either (+)-E-10-OH NT or (−)-E-10-OH NT and tested for antihyperalgesial hour later. The results are presented in FIG. 13. Again, (−)-E-10-OH NT produced significant antihyperalgesia (55±7% AH) while (+)-E-10-OH NT was not antihyperalgesic. The magnitude of the antihyperalgesia observed with (−)-E-10-OH NT was consistent with previous experiments (e.g. Experiment 18).

### Example 20

### Comparison of the Antihyperalgesic Activity of (−)-E-10-OH-NT and (+)-E-10-OH-NT in the Rat FCA-Induced Hyperalgesia Assay at 30 mg/kg and 60 mg/kg ip

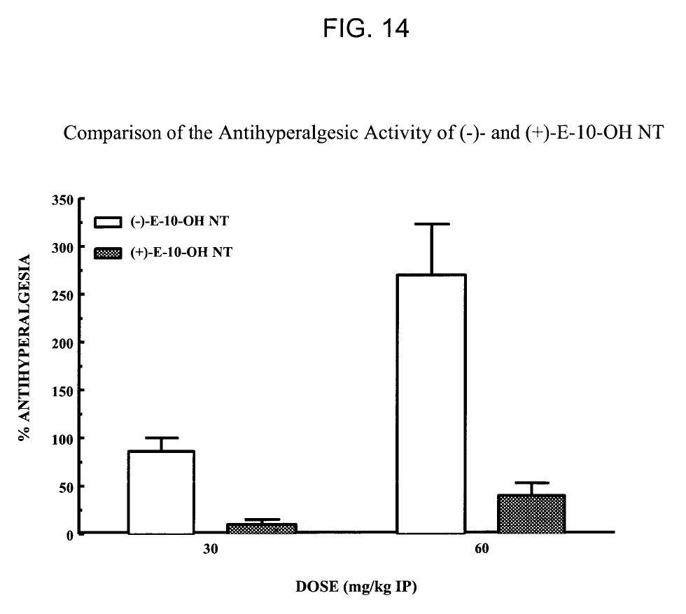

An experiment was performed to extend the dose-response relationship for the effects of (+)-E-10-OH NT in the FCA-induced mechanical hyperalgesia assay (FIG. 14). As demonstrated in Examples 18 and 19, (+)-E-10-OH NT was not active at doses up to 30 mg/kg IP. Accordingly, rats were treated with sterile water vehicle, 30, or 60 mg/kg IP twenty-four hours after administration of FCA. Rats were tested for hyperalgesia 1 hour after treatment with vehicle or (+)-E-10-OH NT.

Results: Statistically significant, but moderate levels of antihyperalgesic activity were observed in rats treated with 60 mg/kg IP (40±13% AH), but not 30 mg/kg IP of (+)-E-10-OH-NT. The (−)-E-10-OH NT enantiomer had greater potency and efficacy than the (+)-E-10-OH NT enantiomer over the dose range tested (compounds were administered as either the maleate or succinate salts; dosage amounts are based on the amount of free base).

### Example 21

### Evaluation of Amitriptyline, (−)-E-10-OH-NT and (+)-E-10-OH-NT in the Rat Forced Swim Test of Depression

Protocol: Male Sprague-Dawley rats (approximately 200 g) are placed in a tank of room temperature water for a fifteen minute practice swim. Every five seconds during the first five minutes of the practice swim, the rats are rated as immobile (floating with motion needed to keep head above the water), swimming (movement across the swim), or climbing (actively trying to climb out of the tank of water, upward directed movements of the forepaws). The percentage of time the rats spent in each of these responses is calculated.

Approximately 24 h after the practice swim, the rats are treated with vehicle or test compound and placed in the tank for a 5 minute swim. As was the case with the practice swim, the rats are rated as immobile, swimming, or climbing during the test swim and the percentage of time spent in each of these responses is calculated. The data are analyzed by one-way ANOVA with post-hoc analysis to compare the behavioral response after vehicle treatment to the behavioral response after drug treatment for each of the three behavioral responses. The level of significance is set at p<0.05.

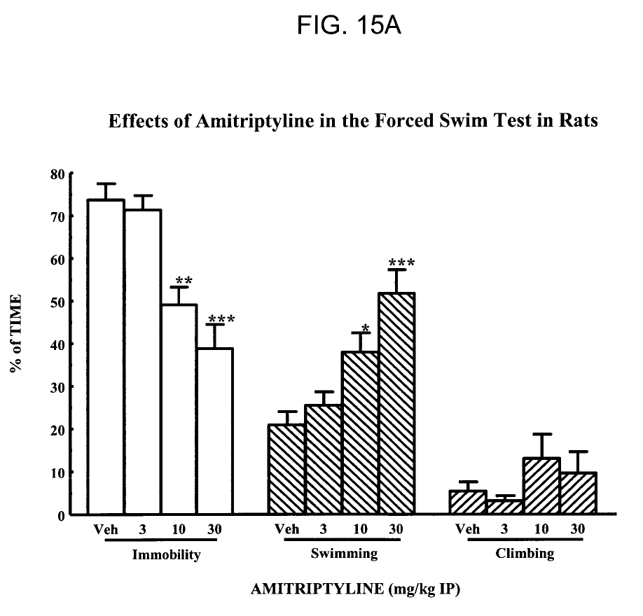

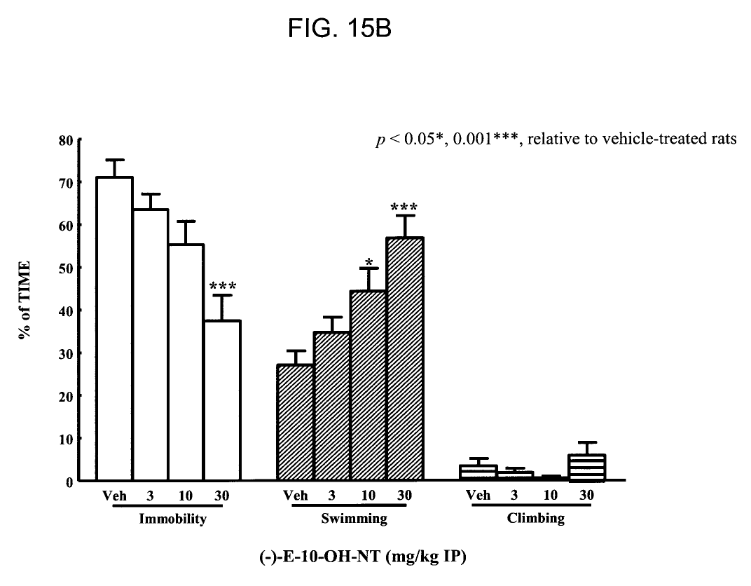

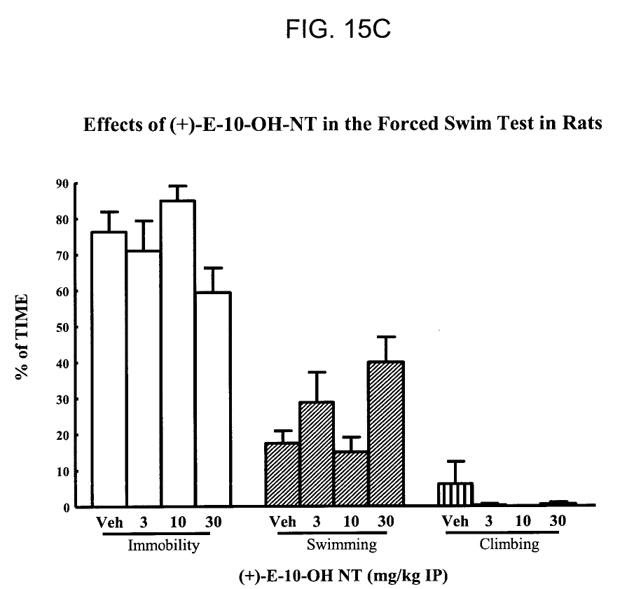

Dose-response experiments were performed with amitriptyline, (−)-E-10-OH NT and (+)-E-10-OH NT in three separate experiments to determine their potencies and efficacies in the forced swim test. In these experiments, 24 hours after a conditioning swim, rats were dosed with vehicle, amitriptyline (3-30 mg/kg IP), (−)-E-10-OH-NT (3-30 mg/kg IP), or (+)-E-10-OH-NT (3-30 mg/kg IP) and 1 h later, the rats were exposed to a 5 minute swim test. The percent of time spent immobile, swimming, and climbing (actively trying to scale the sides of the swim tank) in the test swim is shown in FIG. 15 A, B, and C.

Results: Amitriptyline dose-dependently decreased the amount of time spent immobile, with significant reductions observed in rats treated with 10 or 30 mg/kg IP. Relative to vehicle-treated rats, amitriptyline reduced immobility by 33 and 47% in rats treated with 10 and 30 mg/kg IP, respectively. A corresponding significant increase in the amount of time spent swimming was observed after treatment with 10 mg/kg (81% increase) or 30 mg/kg (147% increase) of amitriptyline. Amitriptyline did not alter the amount of time spent climbing (FIG. 15A).

(−)-E-10-OH-NT produced similar magnitudes of effects as amitriptyline, demonstrating a significant decrease in immobility after treatment with 30 mg/kg IP (47% decrease) and significant increases in swimming in rats treated with 10 (64% increase) or 30 mg/kg IP (108%). As was the case with amitriptyline, (−)-E-10-OH NT did not alter the amount of time spent climbing (FIG. 15B). In contrast, (+)-E-10-OH-NT was not active in the forced swim test at doses up to 30 mg/kg IP (FIG. 15C).

While various specific embodiments have been illustrated and described, it will be appreciated that various changes can be made without departing from the spirit and scope of the invention(s).

All publications, patents, patent applications and other documents cited in this application are hereby incorporated by reference in their entireties for all purposes to the same extent as if each individual publication, patent, patent application or other document were individually indicated to be incorporated by reference for all purposes. The references incorporated herein by reference include:
U.S. Pat. No. 4,943,635
Altamura et al., 1989, International Clinical Psychopharmacology 4(Suppl 1): 103-6.
Asano et al., 2000, Anesthesia & Analgesia (Baltimore) 90(2):400-407.
Atkinson et al., 1999, Pain 83(2):137-145.
Benet et al., 1995, Chapter 1: The Dynamics of Drug Absorption, Distribution, and Elimination, Goodman and Gilman's The Pharmacological Basis of Therapeutics (9^{th} Ed. McGraw Hill).
Berenguer et al., 1994, J. Tetrahedron: Asymmetry 5:165-168.
Bertilsson et al., 1986, Clinical Pharmacology and Therapeutics 40(3):261-267.
Bertrand et al., 1994, Synthetic Communications 24(11):1565-1573.
Bhargava et al., 1996, Neuropeptides 30:219.
Bilsky et al., 1996, J. Pharmacol. Exp. Ther. 277:484.
Buschmann et al., 2002, Analgesics (Wiley-VCH, Verlag GMbH & Co. KgaA, Weinheim)
Bryson & Wilde 1996, Drugs & Aging 8(6):459-476.
Bymaster et al., 2005, Current Pharmaceutical Design 11(12):1475-1493.
Coe et al, 2003, Org. Biomol. Chem. 1: 1106-1111.
Chauret et al., 2001, Drug Metab. Dispos. 29:1196-1200.
Chiral Separation Techniques: A Practical Approach, 2001 (2d ed. Wiley-VCH, Weinheim).
Collins et al., 2000, Journal of Pain and Symptom Management 20(6):449-458.
Corey et al., 1987, J. Am. Chem. Soc. 109:5551-5553.
Corey et al., 1991, J. Org. Chem. 56:442-444.
Corey et al., 1992, J. Am. Chem. Soc. 114:1906-1908.
Corey et al., 1998, Chem., Int. Ed. 37:1986-2012.
Crowell et al., 2004, Current Opinion in Investigational Drugs (Thomson Scientific) 5(7):736-742.
Dahl-Puustinen et al., 1989, Clinical Pharmacology and Therapeutics 45(6):650-656.
DeHaven-Hudkins et al., 1999, J. Pharmacol. Exp. Ther. 289:494-502.
Dennis et al., 1980, Life Science 26:1247-1259.
Deshmukh et al., 2003, Cleveland Clinic Journal of Medicine 70(7):614, 616, 618. Diagnostic and Statistical Manual of Mental Disorders IV, Text Revision, 2000 (American Psychiatric Association, Washington).
Dixon, 1980, Annu Rev Pharmacol Toxicol 20:441-462.
Dourish et al., 1988 Eur. J. Pharmacol. 147:469.
Duquette et al., 2003, Org. Process Res. Dev. 7:285-288.
Edelbroek et al., 1986, Clinical Pharmacology and Therapeutics 39(2):156-62.
Everett et al., 1993, Fundamental and Applied Toxicology 21:59-65.
Fukagawa et al., 1989, American Journal of Physiology 256(3)(Pt. 2):R605-R611.
Goldstein et al., 2005, Pain 116(1-2):109-118.
Gray et al., 1999, European Journal of Pharmacology 378(2):161-168.
Hall et al., 2001, British Journal of Anaesthesia 86(1):5-11.
Hamon & Bourgoin, 2006, European Neuropsychopharmacology 16(Suppl. 5):S625-S632.
Hong et al., 1994 Tetrahedron Lett. 35:6631-6634.
http://www.southcenter.org/publications/pulbichealth/publichealth-07.htm.
Hyttel, 1980, J. Acta Pharmacol. et Toxicol. 47:53-57.
Ingelman-Sundberg et al., 1999, Trends in Pharmacological Sciences 20(8):342-349.
Itsuno et al., 1983, J. Chem. Soc., Chem. Commun. 8:469-470.
Jain, 2000, Emerging Drugs 5(2):241-257.
Kalso et al., 1991, Pharmacology and Toxicology 68:140-143.
Kauppila et al., 1998, Pharmacology Biochemistry and Behavior 59:477-485.
Khawam et al., 2006, Cleveland Clinic Journal of Medicine 73(4):351-3, 356-61.
Kim et al., 2006, Organic Process Research & Development 10(5):949-958.
Kroeze et al., 2003, Neuropsychopharmacology 28(3):519-526.
LaBuda & Little, 2005, J Neurosci Methods 144:175-181.
Lassen et al., 1983, Acta Chemica Scandinavica, Series B: Organic Chemistry and
Biochemistry B37(4):335-340.
Leventhal, et al., 2007, Journal of Pharmacology and Experimental Therapeutics 320(3):1178-1185.
Nichols et al., 1997, Pain 69:317.
Mao et al., 1996, Pain 67:361.
Masaki et al., 2001a, Diabetes 50(2):376-384.
Masaki et al., 2001b, Diabetes 50(2):385-391.
Max et al., 1992, The New England Journal of Medicine 326(19):1250-1256.
Menon et al., Life Sciences 10(19) (Pt. 1):1097-1109.
Millan & Colpaert, 1991, Brain Research 539:342-346.
Nilvebrant et al., 1991, Pharmacology and Toxicology 68(1):64-67.
Nordin et al., 1987a, Clinical Pharmacology and Therapeutics 41(1):97-102.
Nordin et al., 1987b, Clinical Pharmacology and Therapeutics 42(1):10-19.
Nordin et al., 1991, Psychopharmacology 103(3):287-290.
Nordin & Bertilsson, 1995, Clinical Pharmacokinetics 28(1):26-40.
Ongioco et al., 2000, Anesthesiology 92(4):968-976.
Paterson et al., 2005, J. Org. Chem. 70:150-160.
Pearlstein et al., 2003, Journal of Medicinal Chemistry 46(11):2017-2022.
Pertovaara et al., 1990, European Journal of Pharmacology 179:323-328.
Physicians' Desk Reference 1999 (Medical Economics Company).
Quallich et al., 1993, Synlett 12:929-930.
Remington's Pharmaceutical Sciences 2005 (Lippincott Williams & Wilkins).
Remy et al., 1973, Journal of Organic Chemistry 38(4):700-703.
Richelson, 2001, Mayo Clinic Proceedings 76(5):511-527.
Sakai et al., 1995, Behavioral Brain Research 72:83-88.
Sakata et al., 1988, Brain Research 441(1-2):403-407.
Sindrup & Jensen, 1999, Pain 83(3):389-400.
Tagat et al., 2004, J. Med. Chem. 47:2405-2408.
Venhorst et al., 2000, Eur. J. Pharm. Sci. 12:151-158.
Virtanen et al., 1989, Arch. Int. Pharmacodyn. Ther. 297:190-204.
Waegner et al., 1984, Life Sciences 35(13):1379-1383.
Wirshing et al., 1999, Journal of Clinical Psychiatry 60(6):358-363.
Witchel et al., 2002, FEBS Letters 512(1-3):59-66.
Yanagi et al., 2003, Chem. Pharm. Bull. 51:221-223.
Zartman et al., 2005, Bioorg. Med. Chem. Lett. 15:1647-1650.

What is claimed is:

1. A pharmaceutical composition comprising E-10-OH-NT and a pharmaceutically acceptable excipient, carrier and/or diluent, wherein said E-10-OH-NT is enriched in the (−)-enantiomer.

2. The pharmaceutical composition of claim 1, wherein said E-10-OH-NT contains at least about 60% (−)-E-10-OH-NT.

3. The pharmaceutical composition of claim 1, wherein said E-10-OH-NT contains at least about 70% (−)-E-10-OH-NT.

4. The pharmaceutical composition of claim 1, wherein said E-10-OH-NT contains at least about 80% (−)-E-10-OH-NT.

5. The pharmaceutical composition of claim 1, wherein said E-10-OH-NT contains at least about 90% (−)-E-10-OH-NT.

6. The pharmaceutical composition of claim 1, wherein said E-10-OH-NT is substantially enantiomerically pure (−)-E-10-OH-NT.

7. The pharmaceutical composition of claim 1, wherein said E-10-OH-NT is enantiomerically pure (−)-E-10-OH-NT.

8. The pharmaceutical composition of claim 1, wherein said E-10-OH-NT is present in the composition as a salt.

9. The pharmaceutical composition of claim 8 in which the salt is a D- or L-malate salt.

10. The pharmaceutical composition of claim 8 in which the salt is a succinate salt.

11. A method of treating pain in a mammal comprising administering to the mammal an effective amount of a pharmaceutical composition according to any one of claims 1-10.

12. The method of claim 11 in which said pain is of nociceptive origin.

13. The method of claim 11 in which said pain is of non-nociceptive origin.

14. The method of claim 11 in which the pain is neuropathic pain, inflammatory pain, post herpetic neuralgia, diabetic neuropathy or chemo-therapy induced neuropathic pain.

15. The method of claim 11 in which said composition is formulated for oral administration.

16. A method of treating depression in a mammal, comprising administering to the mammal an effective amount of a pharmaceutical according to any one of claims 1-10.

17. A method of treating a disorder in a patient that is responsive to treatment with amitriptyline or nortriptyline, comprising administering to the patient an effective amount of a pharmaceutical composition according to any one of claims 1-10.

18. The method of claim 17 in which said disorder responsive to amitriptyline or nortriptyline therapy is selected from urinary disorders, mood disorders, cognitive disorders, psychotic disorders, anxiety disorders, personality disorders, eating disorders chemical dependencies resulting from addictions to drugs or substances of abuse, withdrawal syndromes, endocrine disorders, impulse disorders, tic disorders, gastrointestinal tract disorders, vascular disorders, somatoform disorders, Parkinson's disease, shock, hypertension, sexual dysfunction, pre-menstrual syndrome and fibromyalgia syndrome.

19. The method of claim 17 in which the patient is taking medication contraindicated for use with amitriptyline or nortriptyline.

20. Substantially enantiomerically pure (−)-E-5-(γ-bromopropylidene)-10,11-dihydro-10-hydroxy-5H-dibenzo[a,d]-cycloheptene.

21. Enantiomerically pure (−)-E-5-(γ-bromopropylidene)-10,11-dihydro-10-hydroxy-5H-dibenzo[a,d]-cycloheptene.

22. Synthetically derived enantiomerically pure (−)-E-10-OH-NT.

23. The synthetically derived enantiomerically pure (−)-E-10-OH-NT of claim 22 in which the E-10-OH-NT is in the form of the free base.

24. The synthetically derived enantiomerically pure (−)-E-10-OH-NT of claim 22 in which the E-10-OH-NT is in the form of a salt.

25. The synthetically derived enantiomerically pure (−)-E-10-OH-NT of claim 22 in which the salt is a malate salt.

26. The synthetically derived enantiomerically pure (−)-E-10-OH-NT of claim 22 in which the salt is a succinate salt.

27. A method of making (−)-E-5-(γ-bromopropylidene)-10,11-dihydro-10-hydroxy-5H-dibenzo[a,d]-cycloheptene, comprising reducing E-5-(γ-bromopropylidene)-10,11-dihydro-10-oxo-5H-dibenzo[a,d]-cycloheptene in the presence of a chiral-specific oxazaborolidine catalyst.

28. The method of claim 27 in which the oxazaborolidine catalyst is

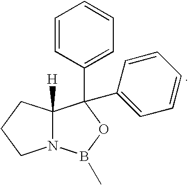

29. A method of making (−)-E-10-OH-NT, comprising reacting (−)-E-5-(γ-bromopropylidene)-10,11-dihydro-10-hydroxy-5H-dibenzo[a,d]-cycloheptene with methylamine.

30. A method of making a substantially enantiomerically pure (−)-E-10-OH-NT, comprising the steps of reacting substantially enantiomerically pure (−)-5-(γ-bromopropylidene)-10,11-dihydro-10-hydroxy-5H-dibenzo[a,d]-cycloheptene with methylamine.

31. A method of making (−)-E-10-OH-NT, comprising the steps of:
(i) reducing E-5-(γ-bromopropylidene)-10,11-dihydro-10-oxo-5H-dibenzo[a,d]-cycloheptene in the presence of 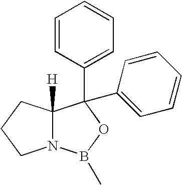
to yield (−)-E-5-(γ-bromopropylidene)-10,11-dihydro-10-hydroxy-5H-dibenzo[a,d]-cycloheptene; and
(ii) reacting the (−)-E-5-(γ-bromopropylidene)-10,11-dihydro-10-hydroxy-5H-dibenzo[a,d]-cycloheptene with methylamine.

32. A method for inhibiting uptake of norepinephrine comprising contacting a norepinephrine transporter with a composition comprising E-10-OH-NT that is enriched in the (−) enantiomer.

33. The method of claim 32 which is practiced in vitro.

34. The method of claim 32 which is practiced in vivo by administering to a subject an amount of a composition comprising an amount of E-10-OH-NT effect to inhibit reuptake of nonepinephrine, wherein said E-10-OH-NT is enriched in the (−) enantiomer.
